(19) United States
(12) Patent Application Publication
(10) Pub. No.: US 2019/0122752 A1
(43) Pub. Date: Apr. 25, 2019

(54) MEDICAL INFORMATION PROCESSING APPARATUS AND MEDICAL INFORMATION PROCESSING METHOD

(71) Applicant: CANON MEDICAL SYSTEMS CORPORATION, Otawara-shi (JP)

(72) Inventors: Kazumasa NORO, Shioya (JP); Kazuhisa MURAKAMI, Tsu (JP); Yusuke KANO, Nasushiobara (JP)

(73) Assignee: CANON MEDICAL SYSTEMS CORPORATION, Otawara-shi (JP)

(21) Appl. No.: 16/167,969

(22) Filed: Oct. 23, 2018

(30) Foreign Application Priority Data
Oct. 24, 2017 (JP) 2017-205308

(51) Int. Cl. G16H 10/60; G06F 17/30; G06F 17/18

(52) U.S. Cl. CPC G16H 10/60; G06F 17/18; G06F 17/30707

(57) ABSTRACT

A medical information processing apparatus according to an embodiment includes processing circuitry. The processing circuitry generates integrated data obtained by integrating information outside a hospitalization period and information during the hospitalization period. The processing circuitry classifies information included in the integrated data into categories based on a period and a type. The processing circuitry calculates an influence degree of the information of the integrated data included in corresponding one of the categories related to a designated item as an analysis object of a patient with respect to the item.

### CROSS-REFERENCE TO RELATED APPLICATIONS

This application is based upon and claims the benefit of priority from Japanese Patent Application No. 2017-205308, filed on Oct. 24, 2017; the entire contents of which are incorporated herein by reference.

### FIELD

Embodiments described herein relate generally to a medical information processing apparatus and a medical information processing method.

### BACKGROUND

In the related art, hospitals and the like have introduced a clinical path defining a standard medical care plan to improve quality of medical care. As a technique for improving the clinical path, there is known a technique of extracting an improvement item of the clinical path by collecting a variance as a difference between the standard medical care plan described in the clinical path and actual medical care, and analyzing a causes of the variance.

### BRIEF DESCRIPTION OF THE DRAWINGS

FIG. 1 is a diagram illustrating an example of a configuration of a medical information processing apparatus according to a first embodiment;

FIG. 2 is a diagram illustrating an example of inspection data acquired by a control function according to the first embodiment;

FIG. 3 is a diagram illustrating an example of operation recording data acquired by the control function according to the first embodiment;

FIG. 4 is a diagram illustrating an example of radiation treatment recording data acquired by the control function according to the first embodiment;

FIG. 5 is a diagram illustrating an example of clinical path master data acquired by the control function according to the first embodiment;

FIG. 6 is a diagram illustrating an example of medical practice/outcome master data acquired by the control function according to the first embodiment;

FIG. 7 is a diagram illustrating an example of clinical path planning data acquired by the control function according to the first embodiment;

FIG. 8 is a diagram illustrating an example of medical practice/outcome detailed master data acquired by the control function according to the first embodiment;

FIG. 9 is a diagram illustrating an example of patient data acquired by the control function according to the first embodiment;

FIG. 10 is a diagram illustrating an example of track record data acquired by the control function according to the first embodiment;

FIG. 11 is a diagram illustrating an example of variance data acquired by an acquisition function according to the first embodiment;

FIG. 12 is a diagram illustrating an example of variance ID master data acquired by the acquisition function according to the first embodiment;

FIG. 13 is a diagram illustrating an example of setting information stored by storage according to the first embodiment;

FIG. 14 is a diagram illustrating an example of the setting information stored by the storage according to the first embodiment;

FIG. 15 is a diagram illustrating an example of the setting information stored by the storage according to the first embodiment;

FIG. 16 is a diagram illustrating an example of the setting information stored by the storage according to the first embodiment;

FIG. 17 is a diagram for explaining an example of processing performed by a data integration function according to the first embodiment;

FIG. 18 is a diagram illustrating an example of data integrated by the data integration function according to the first embodiment;

FIG. 19 is a diagram illustrating an example of integrated data generated by the data integration function according to the first embodiment;

FIG. 20 is a diagram illustrating an example of classification of the integrated data performed by a category classification function according to the first embodiment;

FIG. 21 is a diagram illustrating an example of classification of the integrated data performed by the category classification function according to the first embodiment;

FIG. 22 is a diagram illustrating an example of classification of the integrated data performed by the category classification function according to the first embodiment;

FIG. 23 is a diagram illustrating an example of classification of the integrated data performed by the category classification function according to the first embodiment;

FIG. 24A is a diagram illustrating a modification of classification performed by the category classification function according to the first embodiment;

FIG. 24B is a diagram illustrating a modification of classification performed by the category classification function according to the first embodiment;

FIG. 25 is a diagram illustrating an example of a GUI for designating an analysis object according to the first embodiment;

FIG. 26 is a diagram illustrating an example of converting a condition according to the first embodiment;

FIG. 27A is a diagram illustrating an example of record extraction performed by an influence degree calculation function according to the first embodiment;

FIG. 27B is a diagram illustrating an example of record extraction performed by the influence degree calculation function according to the first embodiment;

FIG. 28A is a diagram illustrating an example of setting of explanatory variables performed by the influence degree calculation function according to the first embodiment;

FIG. 28B is a diagram illustrating an example of setting of response variables performed by the influence degree calculation function according to the first embodiment;

FIG. 29A is a diagram for explaining an example of influence degree calculation performed by the influence degree calculation function according to the first embodiment;

FIG. 29B is a diagram for explaining an example of influence degree calculation performed by the influence degree calculation function according to the first embodiment;

FIG. 30 is a diagram illustrating an example of a calculation result of influence degrees obtained by the influence degree calculation function according to the first embodiment;

FIG. 31 is a diagram illustrating an example of display of influence degrees performed by a display control function according to the first embodiment;

FIG. 32 is a flowchart illustrating a procedure of processing performed by the medical information processing apparatus according to the first embodiment;

FIG. 33 is a flowchart illustrating a procedure of processing performed by the medical information processing apparatus according to the first embodiment;

FIG. 34 is a flowchart illustrating a procedure of processing performed by the medical information processing apparatus according to the first embodiment;

FIG. 35 is a flowchart illustrating a procedure of processing performed by the medical information processing apparatus according to the first embodiment;

FIG. 36 is a diagram illustrating an example of a configuration of a medical information processing apparatus according to a second embodiment;

FIG. 37 is a diagram illustrating an example of compilation of influence degrees performed by an influence degree compiling function according to the second embodiment;

FIG. 38 is a diagram illustrating an example of display of influence degrees performed by a display control function according to the second embodiment;

FIG. 39 is a diagram illustrating an example of display of influence degrees performed by the display control function according to the second embodiment;

FIG. 40 is a diagram illustrating an example of display of influence degrees performed by the display control function according to the second embodiment; and

FIG. 41 is a diagram for explaining analysis content according to a third embodiment.

### DETAILED DESCRIPTION

A medical information processing apparatus according to an embodiment includes a processing circuitry. The processing circuitry is configured to generate integrated data obtained by integrating information outside a hospitalization period and information during the hospitalization period. The processing circuitry is configured to classify information contained in the integrated data into categories based on a period and a type. The processing circuitry is configured to calculate an influence degree of the information of the integrated data included in corresponding one of the categories related to a designated item as an analysis object of a patient with respect to the item.

The following describes embodiments of a medical information processing apparatus and a medical information processing method in detail with reference to the drawings. In the embodiments, information including a series of treatment information such as an inspection result before treatment, a clinical path, an operation, and radiation treatment is described as total treatment information.

### First Embodiment

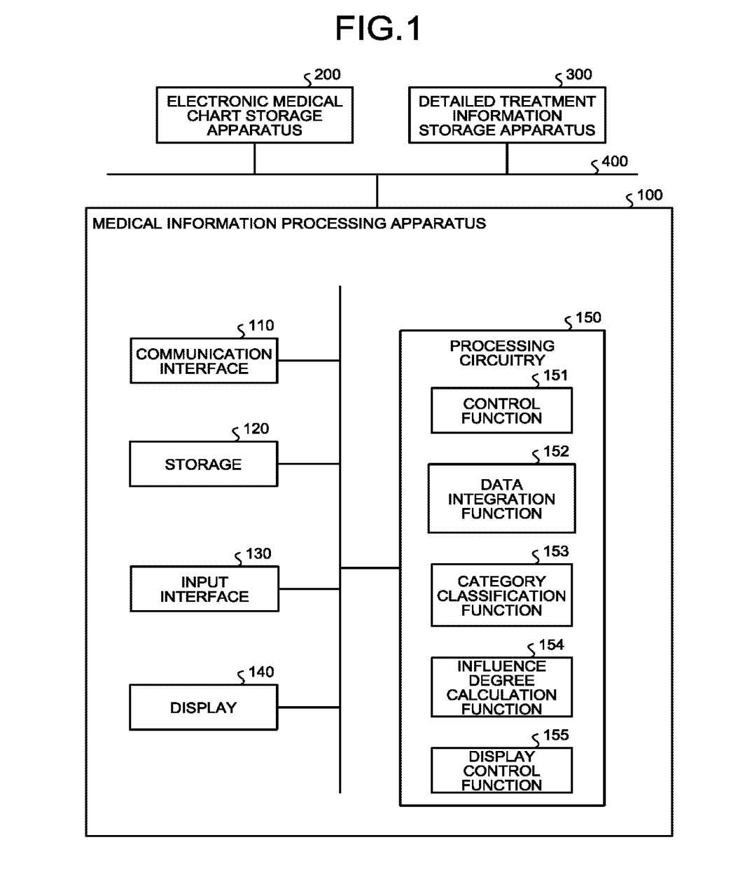

FIG. 1 is a diagram illustrating an example of a configuration of a medical information processing apparatus according to a first embodiment. For example, as illustrated in FIG. 1, a medical information processing apparatus 100 according to the present embodiment is connected to an electronic medical chart storage apparatus 200 and a detailed treatment information storage apparatus 300 in a communicable manner via a network 400. For example, the medical information processing apparatus 100, the electronic medical chart storage apparatus 200, and the detailed treatment information storage apparatus 300 are installed in a hospital and the like, and connected to each other via the network 400 such as an in-hospital LAN and the like. In FIG. 1, only the medical information processing apparatus 100, the electronic medical chart storage apparatus 200, and the detailed treatment information storage apparatus 300 are connected to the network 400, but the embodiment is not limited thereto. Alternatively, other various devices may be connected to the network 400.

The electronic medical chart storage apparatus 200 stores medical care data related to medical care of various kinds provided in a hospital and the like. For example, the electronic medical chart storage apparatus 200 is installed as part of an electronic medical chart system that is introduced into a hospital and the like, and stores medical care data generated by the electronic medical chart system. For example, the electronic medical chart storage apparatus 200 is implemented by a computer appliance such as a database (DB) server, and causes a semiconductor memory element such as a random access memory (RAM) and a flash memory, and storage such as a hard disk and an optical disc to store the medical care data.

The detailed treatment information storage apparatus 300 stores detailed treatment data related to treatment of various kinds performed in a hospital. For example, the detailed treatment information storage apparatus 300 is installed as part of the electronic medical chart system that is introduced into a hospital and the like, and stores the detailed treatment data generated by the electronic medical chart system. For example, the detailed treatment information storage apparatus 300 is implemented by a computer appliance such as a database (DB) server, and causes a semiconductor memory element such as a random access memory (RAM) and a flash memory, and storage such as a hard disk and an optical disc to store the detailed treatment data.

As illustrated in FIG. 1, the medical information processing apparatus 100 includes a communication interface 110, storage 120, an input interface 130, a display 140, and processing circuitry 150. The medical information processing apparatus 100 acquires medical care data from the electronic medical chart storage apparatus 200 via the network 400. The medical information processing apparatus 100 acquires the detailed treatment data from the detailed treatment information storage apparatus 300 via the network 400. The medical information processing apparatus 100 then performs information processing of various kinds using the acquired medical care data and detailed treatment data. For example, the medical information processing apparatus 100 is implemented by a computer appliance such as a workstation. Details about the medical care data and the detailed treatment data will be described later.

The communication interface 110 is connected to the processing circuitry 150, and controls transmission and communication of various pieces of data between the electronic medical chart storage apparatus 200 and the detailed treatment information storage apparatus 300. For example, the communication interface 110 receives the medical care data from the electronic medical chart storage apparatus 200, and outputs the received medical care data to the processing circuitry 150. For example, the communication interface 110 receives the detailed treatment data from the detailed treatment information storage apparatus 300, and outputs the received detailed treatment data to the processing circuitry 150. For example, the communication interface 110 is implemented by a network card, a network adapter, a network interface controller (NIC), and the like.

The storage 120 is connected to the processing circuitry 150, and stores various pieces of data. For example, the storage 120 stores the medical care data received from the electronic medical chart storage apparatus 200, and the detailed treatment data received from the detailed treatment information storage apparatus 300. For example, the storage 120 also stores various pieces of setting information, a processing result obtained by the processing circuitry 150, and the like. For example, the storage 120 is implemented by a semiconductor memory element such as a RAM and a flash memory, a hard disk, an optical disc, and the like.

The input interface 130 is connected to the processing circuitry 150, and converts an input operation received from an operator (user) into an electric signal to be output to the processing circuitry 150. For example, the input interface 130 is implemented by a trackball, a switch button, a mouse, a keyboard, a touch pad including an operation surface to be touched to perform an input operation, a touch screen obtained by integrating a display screen and a touch pad, a noncontact input circuit with an optical sensor, a voice input circuit, and the like.

The display 140 is connected to the processing circuitry 150, and displays various pieces of information output from the processing circuitry 150 and various pieces of image data. For example, the display 140 is implemented by a liquid crystal monitor, a cathode ray tube (CRT) monitor, a touch panel, and the like.

The processing circuitry 150 controls components of the medical information processing apparatus 100 in accordance with the input operation that is received from the user via the input interface 130. For example, the processing circuitry 150 causes the storage 120 to store the detailed treatment data and the medical care data output from the communication interface 110. For example, the processing circuitry 150 reads out the medical care data and the detailed treatment data from the storage 120, and performs processing of various kinds to display a processing result on the display 140. For example, the processing circuitry 150 is implemented by a processor.

The entire configuration of the medical information processing apparatus 100 according to the present embodiment has been described above. With this configuration, the medical information processing apparatus 100 according to the present embodiment enables variances to be analyzed using total information of treatment. Specifically, the medical information processing apparatus 100 acquires information about treatment in a period other than a period to which a clinical path is applied in addition to information about treatment related to the clinical path, integrates and analyzes the information related to the clinical path and the information in the period other than the period to which the clinical path is applied to enable variances to be analyzed using the total information of treatment. In other words, the medical information processing apparatus 100 integrates and analyzes the information during the hospitalization period and the information outside the hospitalization period to enable variances to be analyzed using the total information of treatment. Accordingly, the medical information processing apparatus 100 according to the present embodiment is enabled to make analysis in accordance with various purposes. The following describes details about the medical information processing apparatus 100.

The processing circuitry 150 in the medical information processing apparatus 100 includes a control function 151, a data integration function 152, a category classification function 153, an influence degree calculation function 154, and a display control function 155. The processing circuitry 150 is an example of processing circuitry.

The control function 151 controls processing of various kinds related to communication with another device, and processing of various kinds related to data acquisition from another device. For example, the control function 151 acquires information related to a course of treatment performed on the patient. That is, the control function 151 acquires data related to medical practice that is not related to the clinical path and medical practice that is performed in accordance with the clinical path. The control function 151 acquires data related to a variance generated in the clinical path.

By way of example, the control function 151 acquires the medical care data from the electronic medical chart storage apparatus 200. The control function 151 also acquires the detailed treatment data from the detailed treatment information storage apparatus 300. The medical care data includes, for example, inspection data, clinical path master data, medical practice/outcome master data, clinical path planning data, medical practice/outcome detailed master data, patient data, track record data, variance data, and variance ID master data. The detailed treatment data includes, for example, a case report such as operation recording data and radiation treatment recording data. The control function 151 causes the storage 120 to store the acquired pieces of data.

The inspection data is data in which an inspection result for each patient is stored. The clinical path master data is data in which a name of the path and an ID thereof are stored. The medical practice/outcome master data is data in which medical practice, an outcome (a target state of the patient to be achieved in a specific period), and a medical practice/outcome ID thereof are stored. The clinical path planning data is data in which a path ID of the clinical path, the medical practice/outcome ID, and the number of days scheduled for execution thereof are stored. The medical practice/outcome detailed master data is data in which a specific item name of medical practice/outcome and an item ID thereof are stored. The patient data is data in which basic information of the patient is recorded. The track record data is data in which a history of medical practice performed on the patient, a progression of a patient state, and the like are recorded. The variance data is data generated in a case of deviating from the clinical path, and data in which medical practice or an outcome in which a variance is generated, content of the variance, the number of days, and the like are stored. The variance ID master data is data in which an ID related to a cause of the variance, classification of the variance, and the like are recorded. The operation recording data is data in which a record of an operation performed on the patient is stored. The radiation treatment recording data is data in which a record of radiation treatment performed on the patient is stored.

For example, the control function 151 converts each piece of data acquired from the electronic medical chart storage apparatus 200 or the detailed treatment information storage apparatus 300 into a format optimum for analysis to be stored in the storage 120. Herein, information included in each piece of the data is assumed to be directly obtained from the data stored in the electronic medical chart storage apparatus 200 or the detailed treatment information storage apparatus 300, but the embodiment is not limited thereto. For example, in a case in which the information included in each piece of the data includes information that cannot be directly obtained from the data stored in the electronic medical chart storage apparatus 200 or the detailed treatment information storage apparatus 300, the control function 151 may convert the information using a table for conversion, and may cause the storage 120 to store the information. In this case, the table for conversion is stored in the storage 120 in advance.

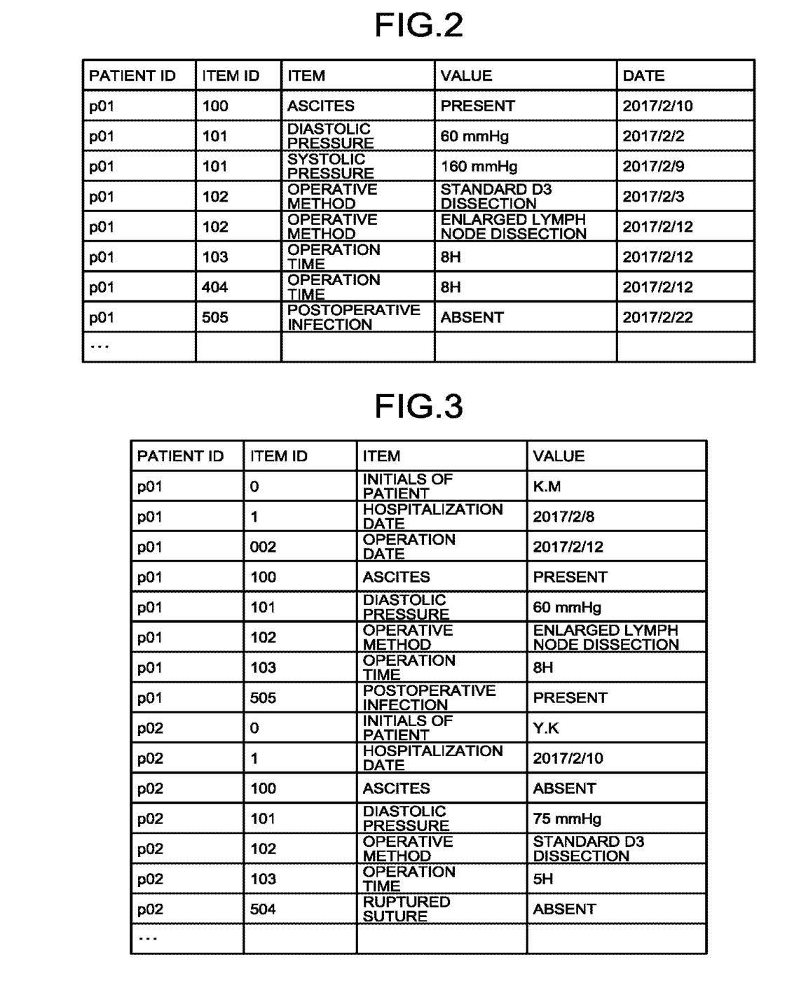

FIG. 2 is a diagram illustrating an example of the inspection data acquired by the control function 151 according to the first embodiment. For example, as illustrated in FIG. 2, the inspection data includes, as data items, a patient ID, an item ID, an item, a value, and a date. As the patient ID, set is an ID for uniquely identifying the patient. As the item ID, set is an ID for uniquely identifying the item. As the item, set is an inspection item. As the value, set is a value of an inspection result. As the date, set is a date at which inspection is performed. As the item ID of the inspection item, set is the same value as the item ID in the medical practice/outcome detailed master data.

FIG. 3 is a diagram illustrating an example of the operation recording data acquired by the control function 151 according to the first embodiment. For example, as illustrated in FIG. 3, the operation recording data includes, as data items, a patient ID, an item ID, an item, and a value. As the item, set is an item of information related to an operation. As the value, set is a value of a corresponding item. As the item ID, set is the same value as the item ID in the medical practice/outcome detailed master data.

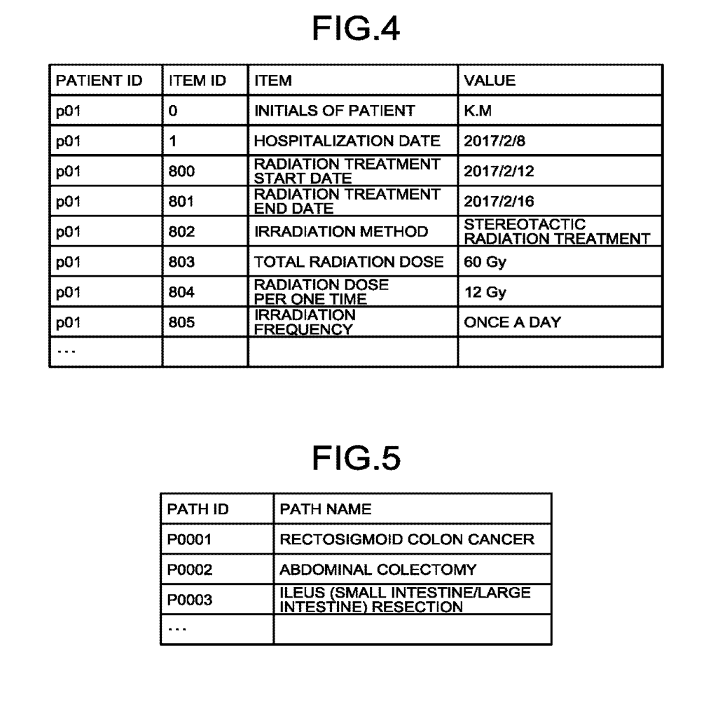

FIG. 4 is a diagram illustrating an example of the radiation treatment recording data acquired by the control function 151 according to the first embodiment. For example, as illustrated in FIG. 4, the radiation treatment recording data includes, as data items, a patient ID, an item ID, an item, and a value. As the item, set is an item of information related to radiation treatment. As the value, set is a value of a corresponding item. As the item ID, set is the same value as the item ID in the medical practice/outcome detailed master data.

FIG. 5 is a diagram illustrating an example of the clinical path master data acquired by the control function 151 according to the first embodiment. For example, as illustrated in FIG. 5, the clinical path master data includes, as data items, a path ID and a path name. As the path ID, set is an ID for uniquely identifying the path. As the path name, set is a name of the path to which the path ID is set.

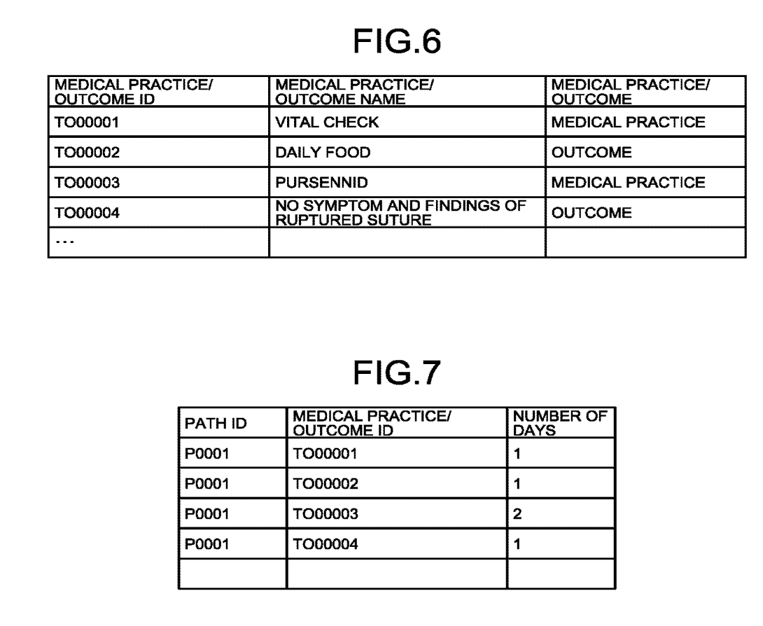

FIG. 6 is a diagram illustrating an example of the medical practice/outcome master data acquired by the control function 151 according to the first embodiment. For example, as illustrated in FIG. 6, the medical practice/outcome master data includes, as data items, a medical practice/outcome ID, a medical practice/outcome name, and a medical practice/outcome. As the medical practice/outcome ID, set is an ID for uniquely identifying the medical practice/outcome. As the medical practice/outcome name, set is a name of the medical practice/outcome to which the medical practice/outcome ID is set. As the medical practice/outcome, set is whether the medical practice/outcome corresponding to the medical practice/outcome ID is medical practice that has been executed or an evaluated outcome. The medical practice includes content and the like related to observation, medication, inspection, treatment, an instruction, nutrition, and explanation that are typically included in the clinical path.

FIG. 7 is a diagram illustrating an example of the clinical path planning data acquired by the control function 151 according to the first embodiment. For example, as illustrated in FIG. 7, the clinical path planning data includes, as data items, a path ID, a medical practice/outcome ID, and the number of days. As the path ID, set is the same value as the path ID in the clinical path master data. As the medical practice/outcome ID, set is the same value as the item ID in the medical practice/outcome master data described above. As the number of days, set is the number of days for which corresponding medical practice/outcome is scheduled to be performed (the number of elapsed days from a clinical path application date (or a hospitalization date)).

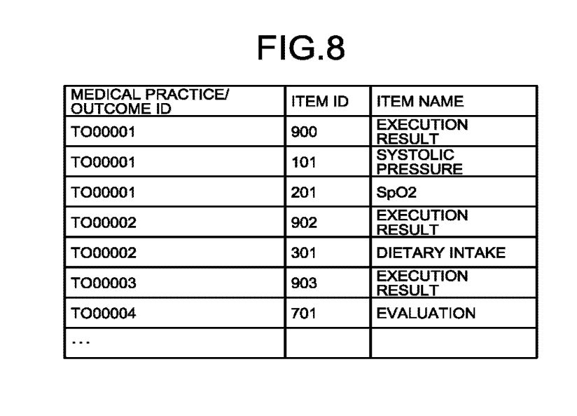

FIG. 8 is a diagram illustrating an example of the medical practice/outcome detailed master data acquired by the control function 151 according to the first embodiment. For example, as illustrated in FIG. 8, the medical practice/outcome detailed master data includes, as data items, a medical practice/outcome ID, an item ID, and an item name. As the medical practice/outcome ID, set is the same value as the medical practice/outcome ID in the medical practice/outcome master data described above. As the item ID, set is an ID for uniquely identifying the item of the medical practice/outcome. As the item name, set is a specific item corresponding to the item ID.

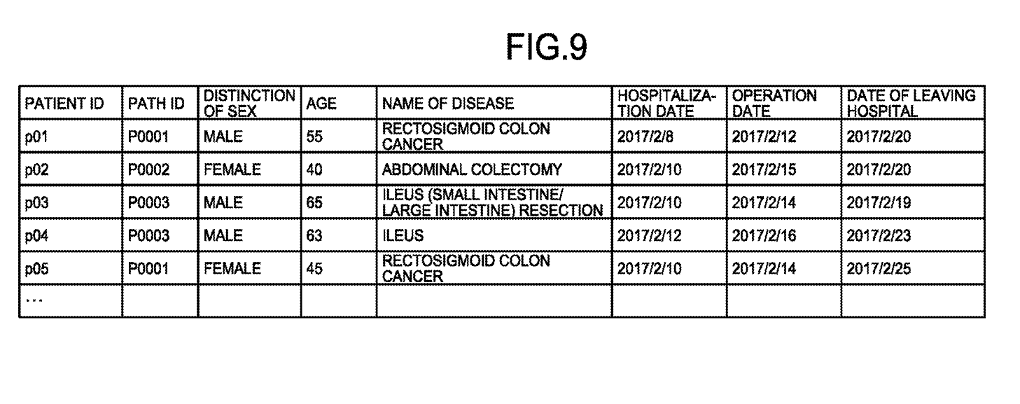

FIG. 9 is a diagram illustrating an example of the patient data acquired by the control function 151 according to the first embodiment. For example, as illustrated in FIG. 9, the patient data includes, as data items, a patient ID, a path ID, a distinction of sex, an age, a name of a disease, a hospitalization date, an operation date, and a date of leaving a hospital. As the patient ID, set is an ID for uniquely identifying the patient, which is the same value as the patient ID in the inspection data and the like described above. As the path ID, set is the same value as the path ID in the clinical path master data described above. As the distinction of sex, set is a distinction of sex of the patient. As the age, set is an age of the patient. As the name of a disease, set is a name of a diagnosed disease of the patient. As the hospitalization date, set is a date at which the patient is hospitalized. As the operation date, set is a date at which an operation is performed on the patient. As the date of leaving a hospital, set is a date at which the patient leaves the hospital.

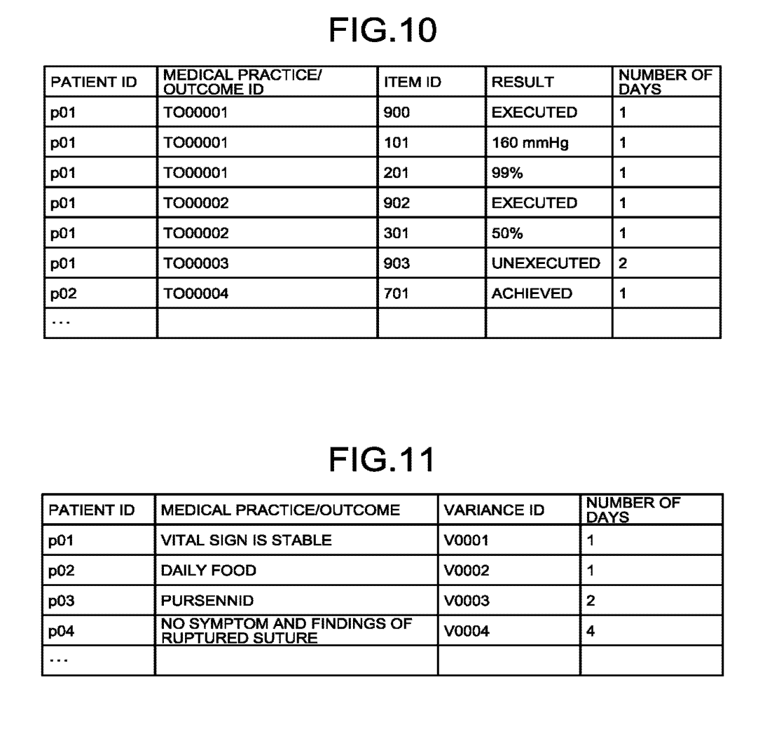

FIG. 10 is a diagram illustrating an example of track record data acquired by the control function 151 according to the first embodiment. For example, as illustrated in FIG. 10, the track record data includes, as data items, a patient ID, a medical practice/outcome ID, an item ID, a result, and the number of days. As the patient ID, set is the same value as the patient ID described above. As the medical practice/outcome ID, set is the same value as the medical practice/outcome ID described above. As the item ID, set is the same value as the item ID in the medical practice/outcome detailed master data described above. As the result, set is a result obtained by evaluating the medical practice or the outcome. As the result, in addition to an execution result of the medical practice (executed/unexecuted), set is data obtained as a result of the medical practice (for example, a vital value and the like obtained as a result of vital check). As the result, set is an evaluation result of the outcome (achieved/unachieved). As the number of days, set is an execution date at which the medical practice or the outcome is evaluated, which indicates the number of days elapsed after the clinical path application date.

FIG. 11 is a diagram illustrating an example of the variance data acquired by the control function 151 according to the first embodiment. For example, as illustrated in FIG. 11, the variance data includes, as data items, a patient ID, a medical practice/outcome, a variance ID, and the number of days. In the variance data, each of the medical practice/outcome, the variance ID, and the number of days is associated with the patient ID to be set. As the patient ID, set is the same value as the patient ID described above. As the medical practice/outcome, set is information indicating the outcome or the medical practice performed on the patient. As the variance ID, set is an ID related to a cause of a variance. As the number of days, set is a generation date at which a variance is generated, which indicates the number of days elapsed after the clinical path application date.

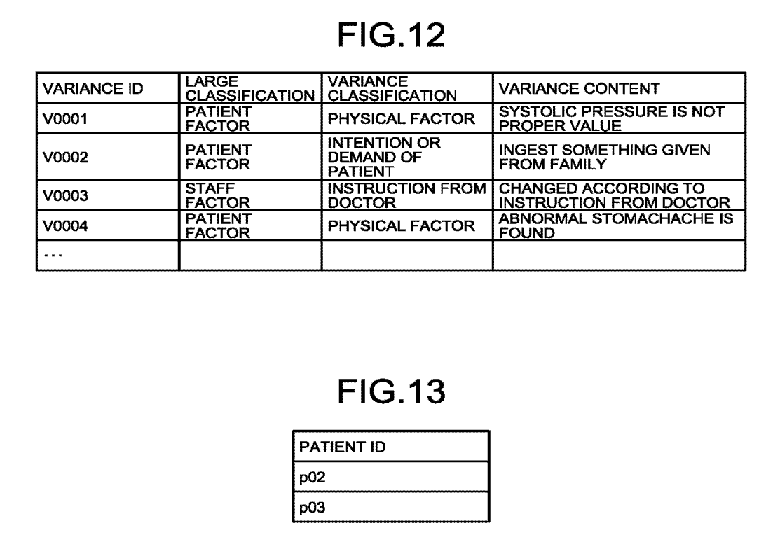

FIG. 12 is a diagram illustrating an example of the variance ID master data acquired by the control function 151 according to the first embodiment. For example, as illustrated in FIG. 12, the variance ID master data includes, as data items, a variance ID, large classification, variance classification, and variance content. As the variance ID, set is the same value as the variance ID in the variance data described above. As the large classification, set is large classification of a cause of the variance (a patient factor, a staff factor, a facility factor, a social factor, and the like). As the variance classification, set is small classification of the cause of the variance (a physical factor, an intention or a demand of the patient, an instruction from a doctor, and the like). As the variance content, set is information indicating content of the variance generated in the clinical path. For example, as the variance content, set is text information describing detailed content of the variance.

The control function 151 acquires the medical care data and the detailed treatment data described above from the electronic medical chart storage apparatus 200 and the detailed treatment information storage apparatus 300, and stores the data in the storage 120. The storage 120 also stores various pieces of setting information in addition to the medical care data and the detailed treatment data described above. Specifically, the storage 120 stores setting information used for processing performed by the processing circuitry 150.

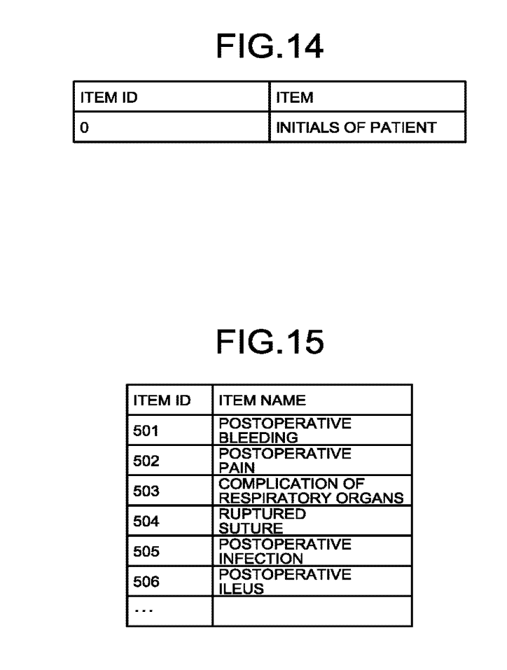

FIGS. 13 to 16 are diagrams illustrating an example of the setting information stored by the storage 120 according to the first embodiment. For example, as illustrated in FIG. 13, the storage 120 stores an exclusion list table (patient). The exclusion list table (patient) includes, as a data item, a patient ID. As the patient ID, set is an ID for uniquely identifying the patient, which is the same value as the patient ID described above. For example, as illustrated in FIG. 14, the storage 120 stores an exclusion list table (item). The exclusion list table (item) includes, as data items, an item ID and an item. As the item ID, set is the same value as the item ID in the medical practice/outcome detailed master data described above. As the item, set is an item corresponding to the item ID. The exclusion list table described above is information used by the data integration function 152 in which content to be excluded in integrating the data is set.

For example, as illustrated in FIG. 15, the storage 120 stores a treatment result master table. The treatment result master table includes, as data items, an item ID and an item name. As the item ID, set is the same value as the item ID in the medical practice/outcome detailed master data described above. As the item name, set is the same value as the item name in the medical practice/outcome detailed master data described above. The treatment result master table described above is information used by the category classification function 153 in which item names as treatment results among various items are set.

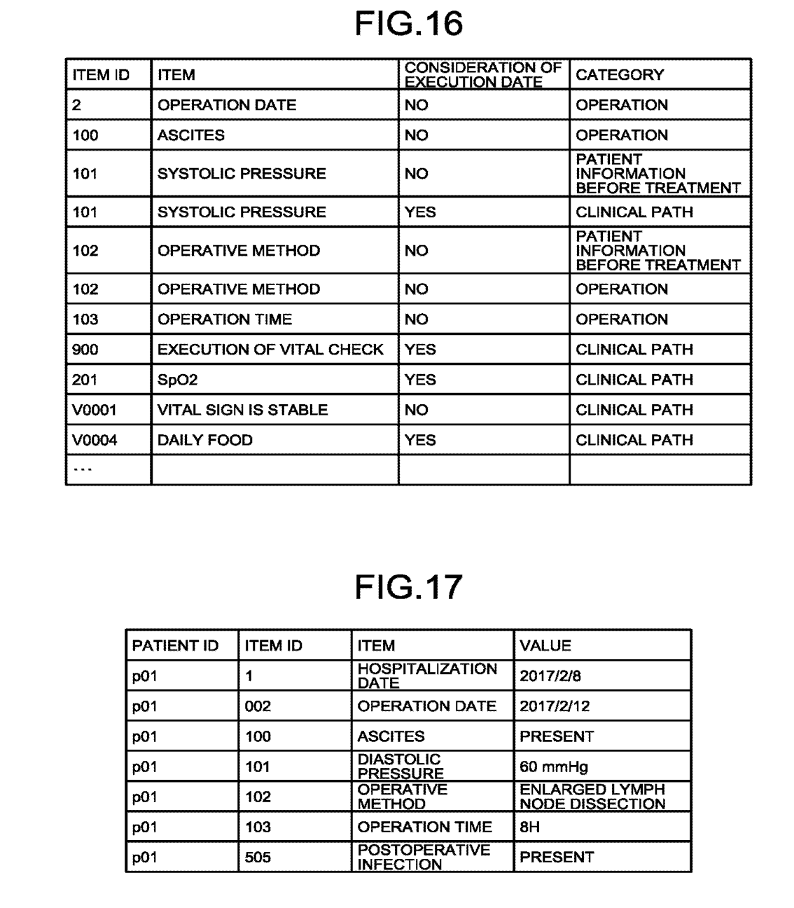

For example, as illustrated in FIG. 16, the storage 120 stores an influence degree calculation setting table. The influence degree calculation setting table includes, as data items, an item ID, an item, consideration of execution date, and a category. As the item ID, set is the same value as the item ID in the medical practice/outcome detailed master data described above. As the item, set is an item corresponding to the item ID. As the consideration of execution date, set is information about whether to consider the execution date at which content of the item is executed in calculating the influence degree. As the category, set is a category including the item. The influence degree calculation setting table described above is information used by the influence degree calculation function 154.

The various pieces of setting information described above can be appropriately edited by the user. For example, the display control function 155 causes the display 140 to display a GUI for editing the setting information, and the user edits the setting information into desired information via the input interface 130.

Returning to FIG. 1, the data integration function 152 generates integrated data obtained by integrating information before and after the period to which the clinical path is applied and information during the period to which the clinical path is applied. Specifically, the data integration function 152 generates the integrated data obtained by integrating information associated with the clinical path and information unassociated with the clinical path. That is, the data integration function 152 generates the integrated data indicating total treatment information of the patient.

For example, the data integration function 152 acquires clinical information used for analysis from the storage 120. By way of example, the data integration function 152 acquires the operation recording data and the inspection data to be integrated. The data integration function 152 generates, as the integrated data, data excluding content included in the exclusion list table stored by the storage 120. FIG. 17 is a diagram for explaining an example of processing performed by the data integration function 152 according to the first embodiment. FIG. 17 illustrates a case of acquiring the data from the operation recording data illustrated in FIG. 3.

For example, the data integration function 152 acquires, as the integrated data, data obtained by excluding content included in the exclusion list table (patient) illustrated in FIG. 13 and the exclusion list table (item) illustrated in FIG. 14 from the operation recording data. That is, as illustrated in FIG. 17, the data integration function 152 acquires data obtained by excluding the patient IDs illustrated in FIG. 13 and the item illustrated in FIG. 14 from the operation recording data illustrated in FIG. 3. In the example described above, a patient and an item to be excluded are excluded based on the exclusion list table, but the embodiment is not limited thereto. All patients and items may be used without exclusion.

The data integration function 152 then integrates the acquired operation recording data and the inspection data stored by the storage 120. The data integration function 152 generates the integrated data that is integrated based on an application date of the clinical path (for example, a hospitalization date). For example, the data integration function 152 generates the integrated data based on “hospitalization date: 2017/2/8” illustrated in FIG. 17. The data integration function 152 acquires the inspection data using, as object data, data in a predetermined period before or after the period to which the clinical path is applied.

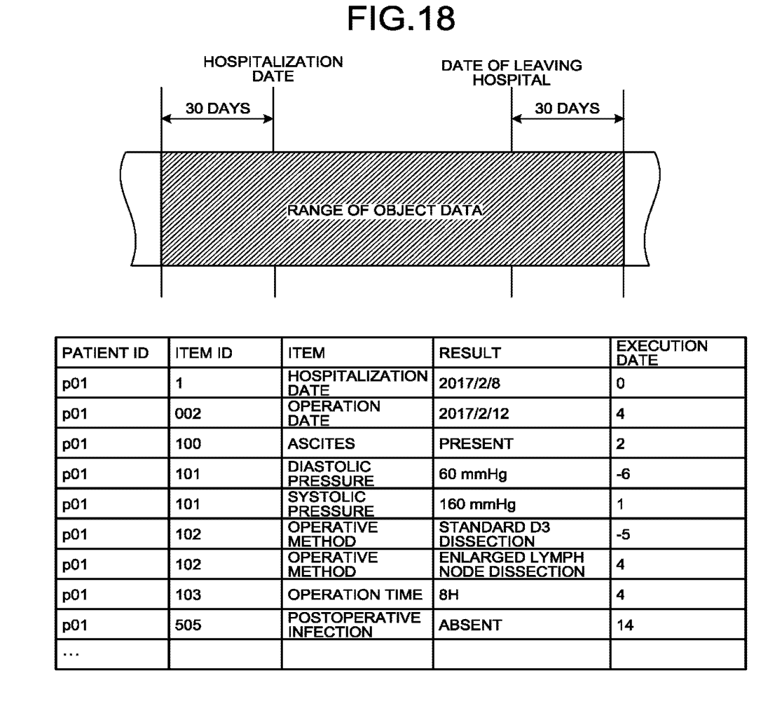

FIG. 18 is a diagram illustrating an example of data integrated by the data integration function 152 according to the first embodiment. For example, as illustrated in an upper row of FIG. 18, the data integration function 152 sets, as a range of the object data, a range from “30 days before the hospitalization date” to “30 days after the date of leaving a hospital”, and acquires, as the object data, data within the range from dates included in the inspection data. For example, as illustrated in FIG. 17, the data integration function 152 acquires the inspection data in a range from 30 days before “hospitalization date: 2017/2/8” to 30 days after “date of leaving a hospital: 2017/2/20” as the object data related to the patient of “patient ID: p01”.

As illustrated in a lower table in FIG. 18, the data integration function 152 generates the integrated data obtained by integrating the operation recording data and the inspection data based on “hospitalization date: 2017/2/8”. That is, as illustrated in FIG. 18, the data integration function 152 generates, assuming that “hospitalization date: 2017/2/8” is “execution date: 0”, the integrated data obtained by adding “execution date” based on “hospitalization date: 2017/2/8” to each item.

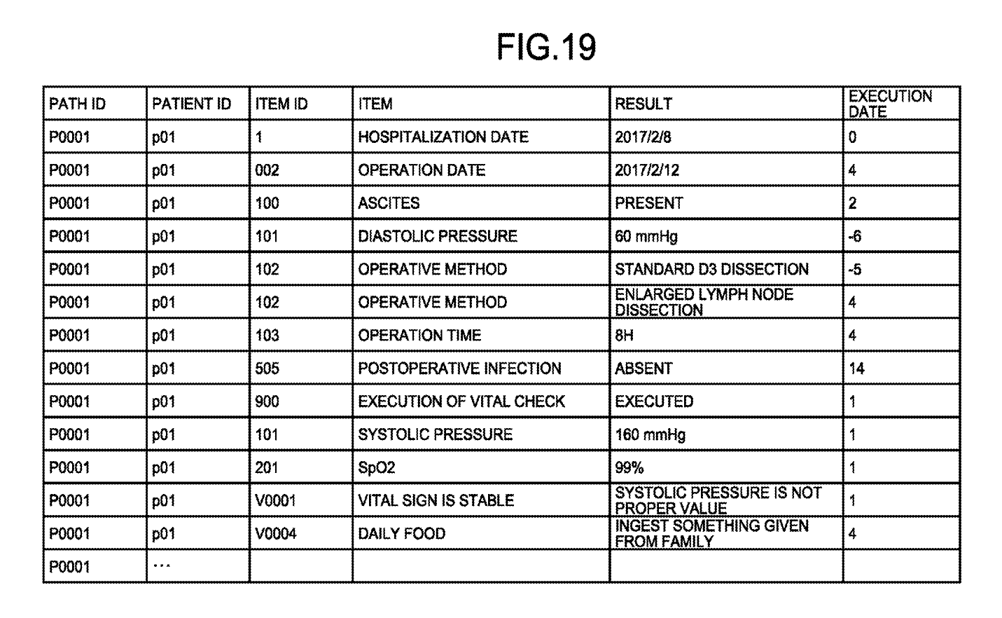

The data integration function 152 further integrates, by using the patient ID and the hospitalization date, the variance data (for example, refer to FIG. 11) and the track record data (for example, refer to FIG. 10) associated with the clinical path into the integrated data. FIG. 19 is a diagram illustrating an example of the integrated data generated by the data integration function 152 according to the first embodiment. For example, as illustrated in FIG. 19, the data integration function 152 generates the integrated data including, as data items, a path ID, a patient ID, an item ID, an item, a result, and an execution date. The data integration function 152 determines whether to cause each item to be included in the integrated data based on the number of days (the number of elapsed days from the clinical path application date) included in the track record data and the variance data. That is, among the items of the track record data and the variance data, the data integration function 152 integrates, into the integrated data, an item having a corresponding number of days included in the range of the object data described above.

In a case in which the track record data already includes an item in which both of the item ID and the execution date are overlapped with those in the clinical path, the data integration function 152 excludes the overlapped record from the object data. For example, the track record data illustrated in FIG. 10 includes a record in which the item ID and the number of days (execution date) are overlapped with “patient ID: p01, item ID: 101, item: systolic pressure, result: 160 mmHg, execution date: 1” illustrated in FIG. 18, so that the data integration function 152 excludes one of the records from the integrated data.

As described above, the data integration function 152 generates the integrated data from the operation recording data, the inspection data, the track record data, and the variance data. The data integration function 152 generates the integrated data described above for each patient to be an object, and stores the generated integrated data in the storage 120.

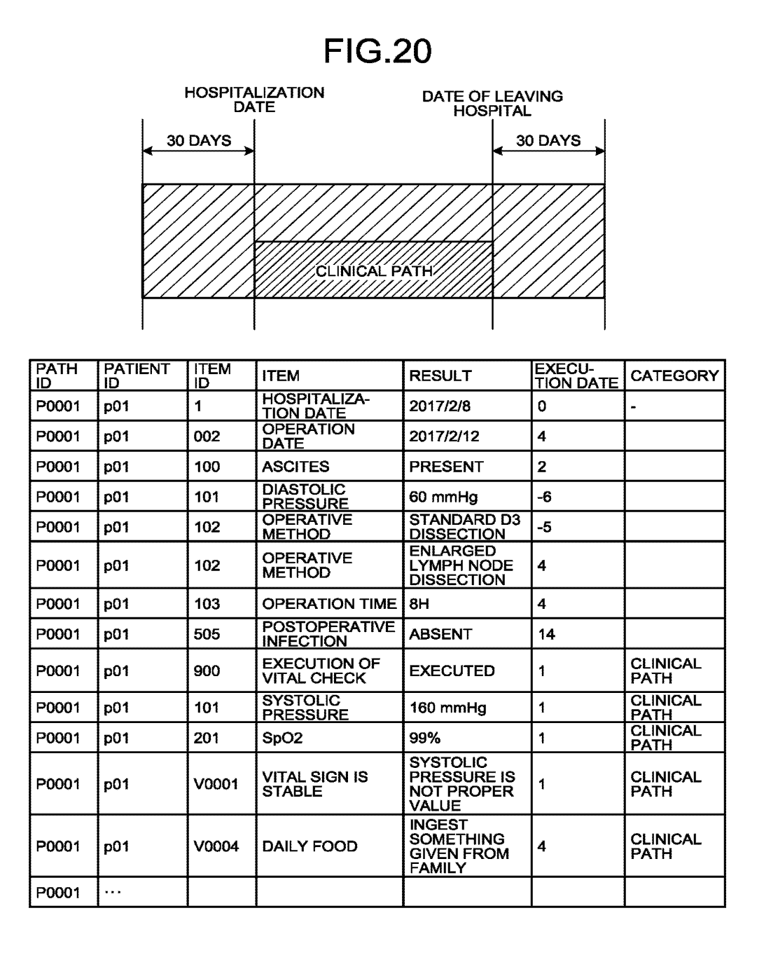

Returning to FIG. 1, the category classification function 153 classifies the information included in the integrated data into a plurality of categories based on a corresponding period and type. Specifically, the category classification function 153 classifies the integrated data stored by the storage 120 into a plurality of categories. For example, the category classification function 153 classifies the information included in the integrated data into categories of patient information before treatment, information about an operation, information about the clinical path, and information about a treatment result. The following describes classification of the integrated data with reference to FIGS. 20 to 23. FIGS. 20 to 23 are diagrams illustrating an example of classification of the integrated data performed by the category classification function 153 according to the first embodiment.

For example, regarding the integrated data illustrated in FIG. 19, the category classification function 153 classifies the integrated item from the track record data and the variance data into “category: clinical path”. That is, as illustrated in FIG. 20, the category classification function 153 classifies, into “category: clinical path”, a record associated with the clinical path in a period from the hospitalization date to the date of leaving a hospital. By way of example, as illustrated in FIG. 20, the category classification function 153 classifies, into “category: clinical path”, a record of “path ID: P0001, patient ID: p01, item ID: 900, item: execution of vital check, result: executed, execution date: 1” in the integrated data illustrated in FIG. 19. Similarly, the category classification function 153 classifies, into “category: clinical path”, each record integrated from the track record data and the variance data.

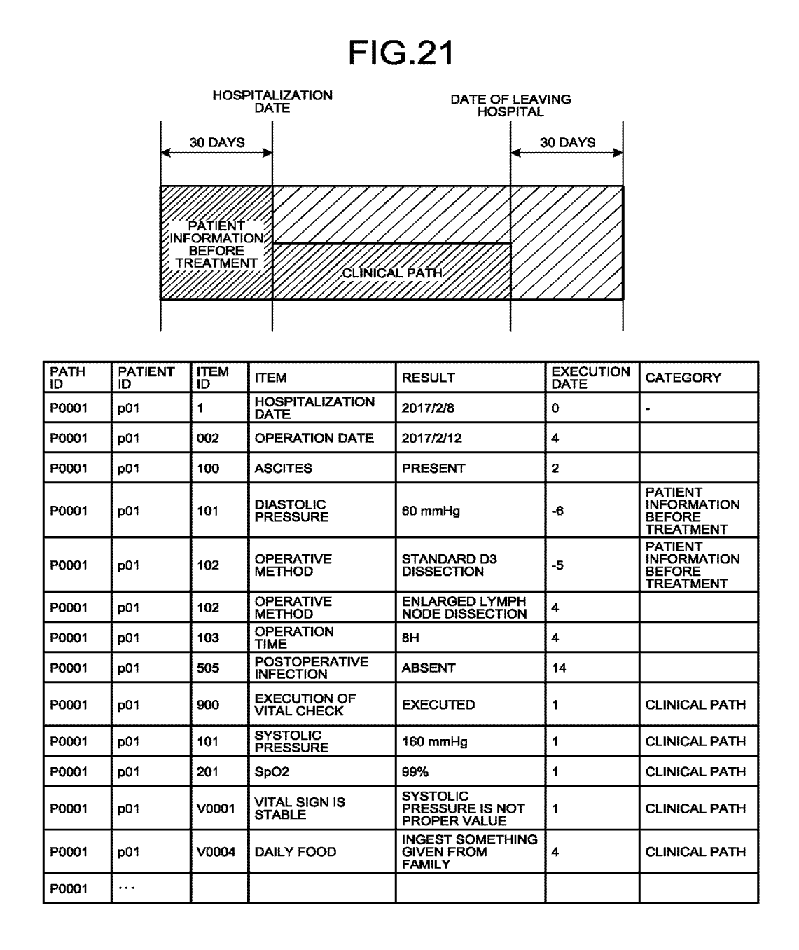

For example, regarding the integrated data illustrated in FIG. 19, the category classification function 153 classifies, into “category: patient information before treatment”, a record in which the execution date is earlier than the hospitalization date. That is, as illustrated in FIG. 21, the category classification function 153 classifies a record in a period from 30 days before the hospitalization date until the hospitalization date into “category: patient information before treatment”. By way of example, as illustrated in FIG. 21, the category classification function 153 classifies, into “category: patient information before treatment”, a record of “path ID: 0001, patient ID: p01, item ID: 101, item: diastolic pressure, result: 60 mmHg, execution date: −6” in the integrated data illustrated in FIG. 19. Similarly, the category classification function 153 classifies a record in a period from 30 days before the hospitalization date until the hospitalization date into “category: patient information before treatment”.

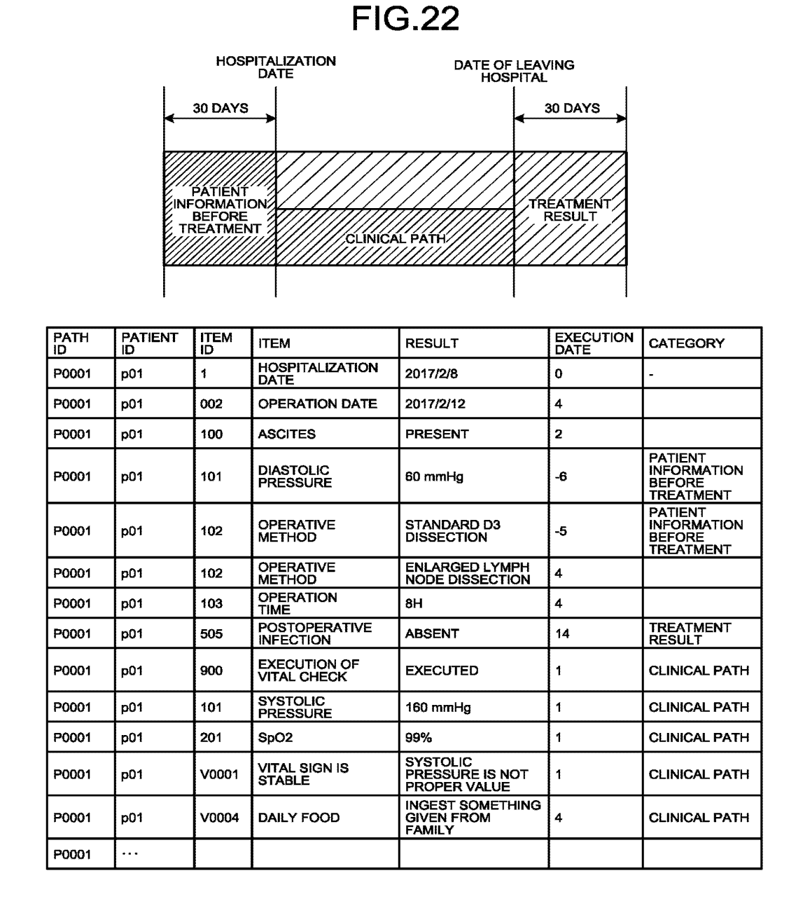

For example, regarding the integrated data illustrated in FIG. 19, the category classification function 153 classifies, into “category: treatment result”, a record including an item ID corresponding to the item ID included in the treatment result master data. For example, as illustrated in FIG. 22, the category classification function 153 refers to the treatment result master data (for example, FIG. 15), and classifies, into “category: treatment result”, a record of “path ID: 0001, patient ID: p01, item ID: 505, item: postoperative infection, result: no, execution date: 14” corresponding to “item ID: 505, item name: postoperative infection” included in the treatment result master data. Similarly, the category classification function 153 classifies, into “category: treatment result”, a record including an item ID corresponding to the item ID included in the treatment result master data. For example, “category: treatment result” is included in a record in a period from the date of leaving a hospital until 30 days after the date of leaving a hospital.

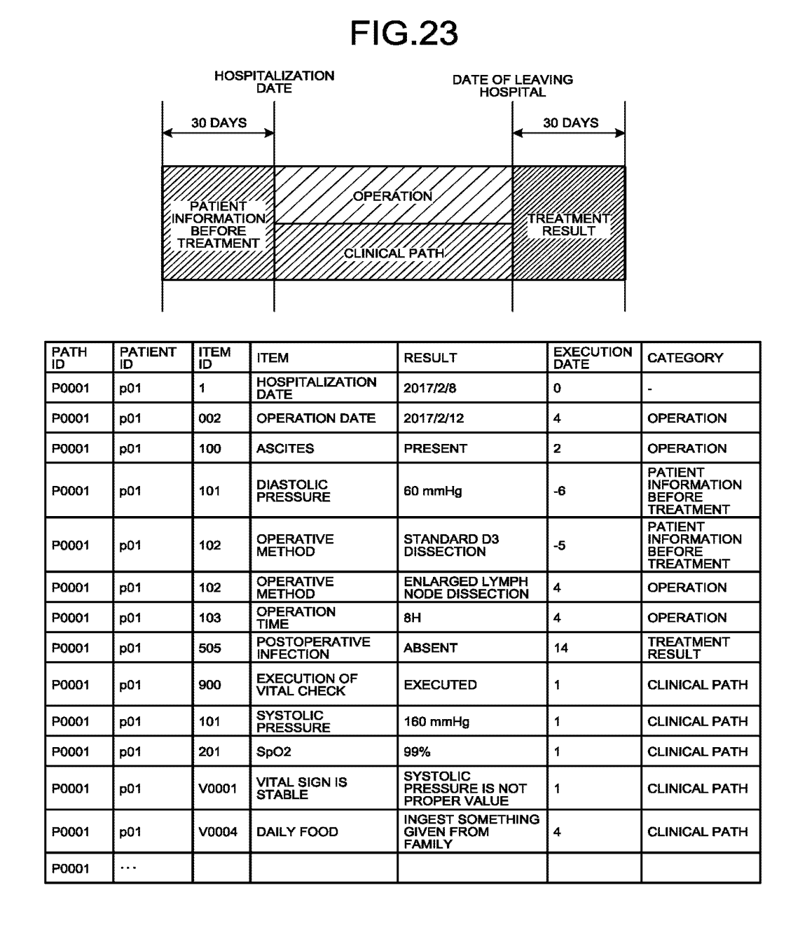

For example, regarding the integrated data illustrated in FIG. 19, the category classification function 153 classifies, into “category: operation”, a record integrated from the operation recording data in which the execution date is in a period from the hospitalization date until the date of leaving a hospital. That is, as illustrated in FIG. 23, the category classification function 153 classifies, into “category: operation”, a record associated with an operation in a period from the hospitalization date until the date of leaving a hospital. By way of example, as illustrated in FIG. 23, the category classification function 153 classifies, into “category: operation”, a record of “path ID: 0001, patient ID: p01, item ID: 002, item: operation date, result: 2017/2/12, execution date: 4” in the integrated data illustrated in FIG. 19. Similarly, the category classification function 153 classifies, into “category: operation”, a record integrated from the operation recording data in which the execution date is in a period from the hospitalization date until the date of leaving the hospital.

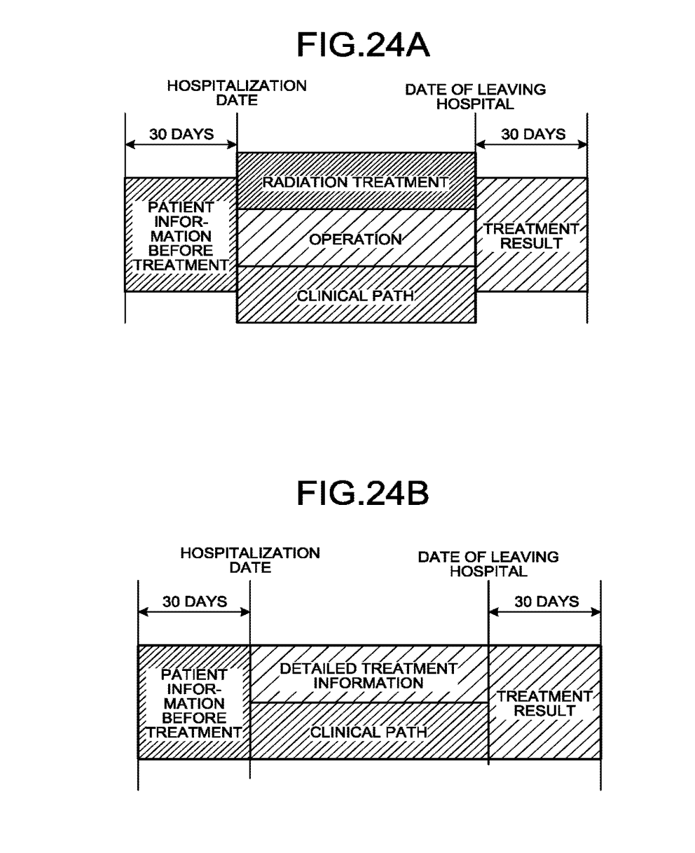

In the embodiment described above, described is a case of classifying the category into four categories of “clinical path”, “patient information before treatment”, “treatment result”, and “operation”. However, the embodiment is not limited thereto, and classification into other categories may be performed. FIGS. 24A and 24B are diagrams illustrating a modification of classification performed by the category classification function 153 according to the first embodiment. For example, as illustrated in FIG. 24A, the category classification function 153 may classify the category into five categories of “clinical path”, “patient information before treatment”, “treatment result”, “operation”, and “radiation treatment”. Alternatively, as illustrated in FIG. 24B, the category classification function 153 may classify the category into four categories of “clinical path”, “patient information before treatment”, “treatment result”, and “detailed treatment information” obtained by combining “operation” with “radiation treatment”.

In such a case, the data integration function 152 generates the integrated data with which the radiation treatment recording data (for example, refer to FIG. 4) is further integrated. For example, similarly to the integration of the operation recording data, the data integration function 152 integrates the radiation treatment recording data into the integrated data using the patient ID and the item ID. The category classification function 153 classifies a record integrated from the operation recording data into “operation”, and classifies a record integrated from the radiation treatment recording data into “radiation treatment”.

Returning to FIG. 1, the influence degree calculation function 154 calculates the influence degree of each piece of information included in a plurality of categories with respect to a designated item as an analysis object in the information included in the integrated data. Specifically, the influence degree calculation function 154 calculates the influence degree of each item included in each category with respect to the information as an analysis object received via the input interface 130. As a method of designating the analysis object as an object of influence degree calculation, various methods can be used. For example, the display control function 155 displays a GUI for designating the analysis object, and the input interface 130 receives an operation of designating the analysis object via the GUI.

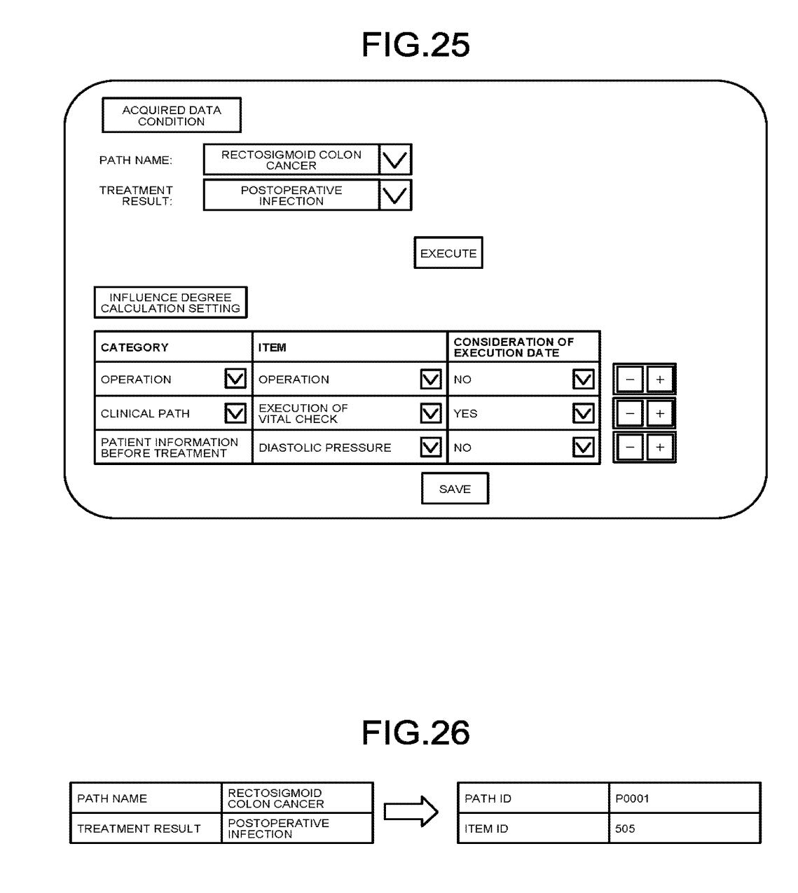

FIG. 25 is a diagram illustrating an example of the GUI for designating the analysis object according to the first embodiment. For example, as illustrated in FIG. 25, the display control function 155 causes the display 140 to display the GUI for receiving an input of “path name” and “treatment result” as “acquired data condition”, and receiving “category”, “item”, and “consideration of execution date” as “influence degree calculation setting”. For example, the input interface 130 receives an input of “rectosigmoid colon cancer” as a path name of the clinical path as an analysis object (from which the data is acquired), and receives an input of “postoperative infection” as a treatment result of the path. The input interface 130 also receives designation of the category, the item, and consideration of execution date as an object the influence degree of which is calculated. When the input interface 130 receives these inputs, the received pieces of information are stored in the storage 120.

The influence degree calculation function 154 calculates the influence degree of the item based on the information stored in the storage 120. For example, when “path name: rectosigmoid colon cancer” and “treatment result: postoperative infection” are input as “acquired data conditions”, the influence degree calculation function 154 acquires the conditions, and refers to pieces of master data to be converted into IDs corresponding to the acquired conditions. That is, the influence degree calculation function 154 converts the conditions into information that can be searched for in the integrated data. For example, the influence degree calculation function 154 refers to the clinical path master data (for example, FIG. 5) and the treatment result master table (for example, FIG. 15), and converts “path name: rectosigmoid colon cancer” and “treatment result: postoperative infection” into “path ID: P0001” and “item ID: 505”, respectively, as illustrated in FIG. 26. FIG. 26 is a diagram illustrating an example of converting the condition according to the first embodiment.

Next, the influence degree calculation function 154 extracts a record for calculating the influence degree from the integrated data classified into categories by the category classification function 153. Specifically, the influence degree calculation function 154 extracts a record corresponding to the ID from the integrated data using the converted ID. For example, from “path ID: 0001” of the integrated data (for example, refer to FIG. 23) that has been classified into categories, the influence degree calculation function 154 extracts a record corresponding to the received treatment result and a record corresponding to the received category.

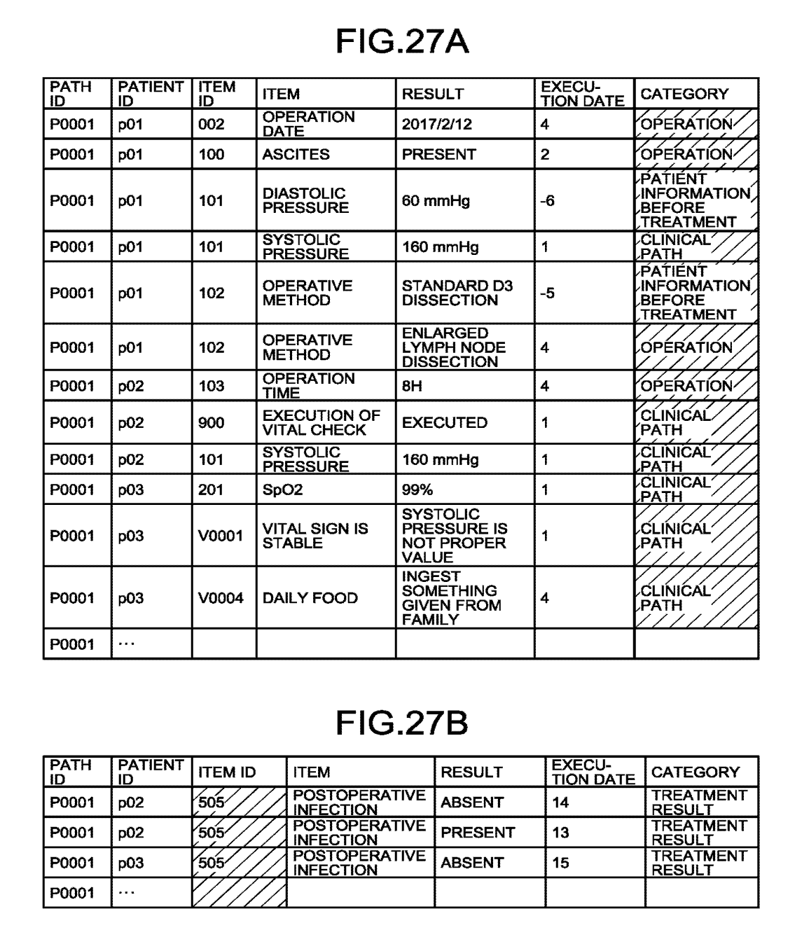

FIGS. 27A and 27B are diagrams illustrating an example of record extraction performed by the influence degree calculation function 154 according to the first embodiment. For example, as illustrated in FIG. 27A, the influence degree calculation function 154 extracts records the categories of which are “operation”, “patient information before treatment”, and “clinical path” from records of “path ID: P0001”. For example, as illustrated in FIG. 27B, the influence degree calculation function 154 extracts a record of “item ID: 505” from records of “path ID: P0001”.

The influence degree calculation function 154 then sets, as explanatory variables, the records the categories of which are “operation”, “patient information before treatment”, and “clinical path” among the extracted records, and sets, as a response variable, the record the category of which is “treatment result”, that is, the record of “postoperative infection” designated as a treatment result. In other words, the influence degree calculation function 154 calculates the influence degree of each item included in the explanatory variable with respect to the treatment result (for example, postoperative infection) set as the response variable.

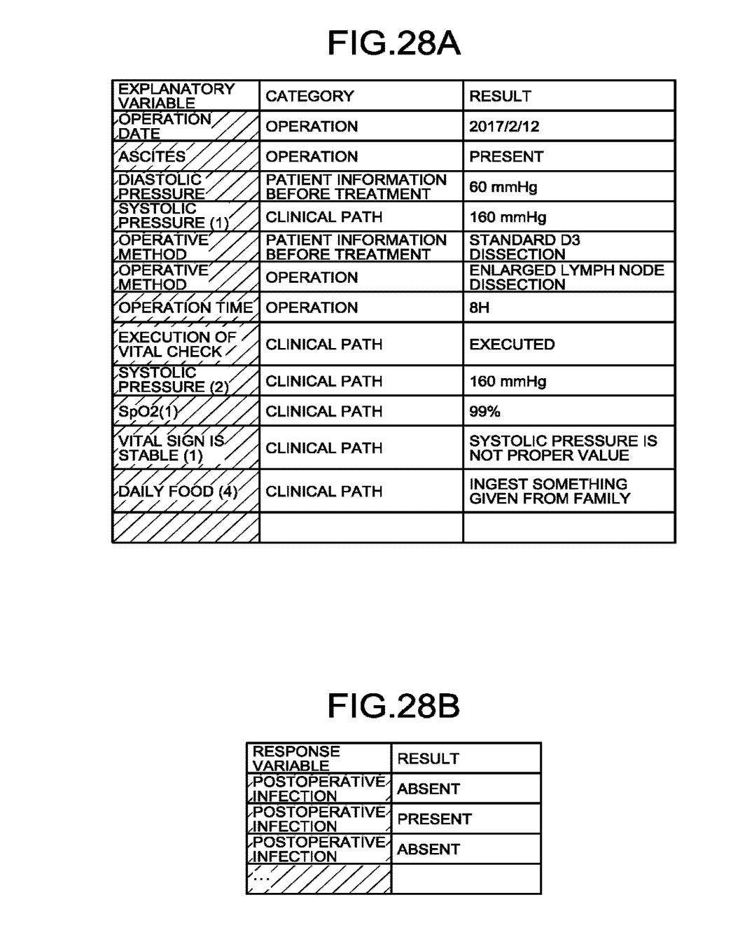

FIG. 28A is a diagram illustrating an example of setting of the explanatory variables performed by the influence degree calculation function 154 according to the first embodiment. FIG. 28B is a diagram illustrating an example of setting of the response variables performed by the influence degree calculation function 154 according to the first embodiment. For example, as illustrated in FIG. 28A, the influence degree calculation function 154 sets, as the explanatory variables, items in the records the categories of which are “operation”, “patient information before treatment”, and “clinical path” (for example, refer to FIG. 27A). For example, as illustrated in FIG. 28B, the influence degree calculation function 154 sets, as the response variable, “postoperative infection” in the records of “item ID: 505” (for example, refer to FIG. 27B). That is, the influence degree calculation function 154 calculates the influence degree of each item of the explanatory variables with respect to the response variable “postoperative infection”.

The influence degree calculation function 154 may also calculate the influence degree separately for each execution date of the items. For example, systolic pressures measured on the first day and the second day are caused to be different items, that is, a systolic pressure (1) and a systolic pressure (2). On the other hand, there are some items the execution date of which is not required to be considered such as an operation time and an operative method. The influence degree calculation function 154 determines whether to consider the execution date of each item with reference to the influence degree calculation setting table described above. In this way, by discriminating the same item based on the execution date, analysis can be made more correctly.

Next, the influence degree calculation function 154 calculates the influence degree using all combinations of the response variables and the explanatory variables. For example, the influence degree calculation function 154 calculates the influence degree using a correlation ratio, a Pearson correlation coefficient, a Cramer's coefficient of association, and the like. In a case in which a result is a numerical value, the influence degree calculation function 154 uses the numerical value as it is for correlation calculation, and in a case in which the result is character data such as “Yes/No”, the influence degree calculation function 154 numbers the data like “0/1” to be used for correlation calculation.

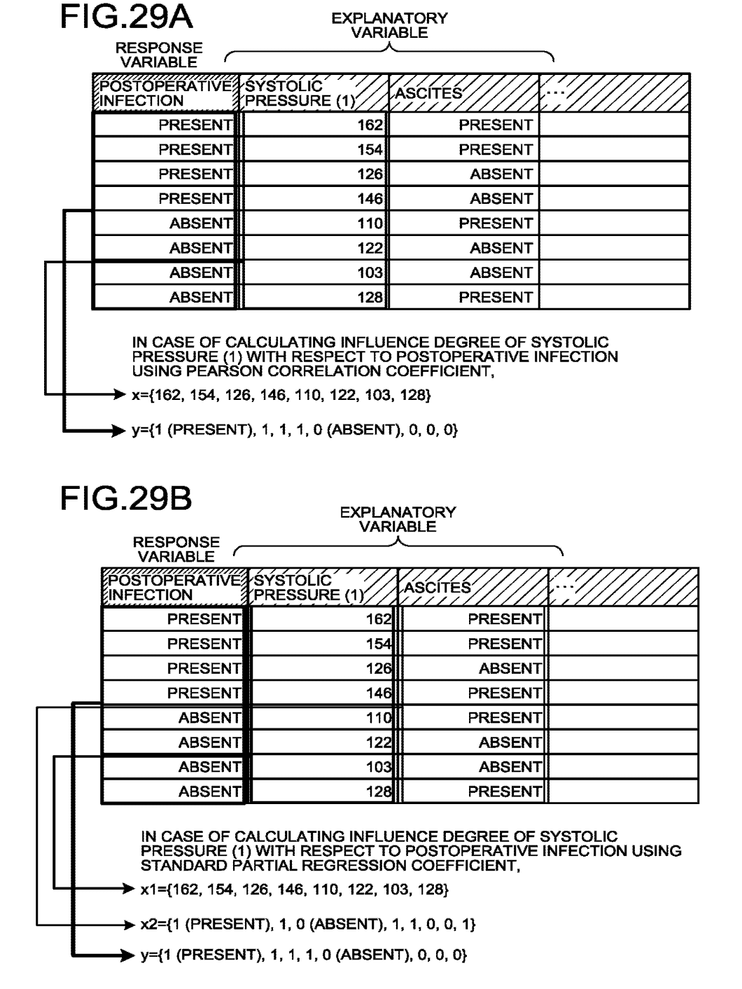

FIGS. 29A and 29B are diagrams for explaining an example of influence degree calculation performed by the influence degree calculation function 154 according to the first embodiment. FIG. 29A illustrates a calculation example in a case of calculating the influence degree using a Pearson correlation coefficient. FIG. 29B illustrates a calculation example in a case of calculating the influence degree using a standard partial regression coefficient.

For example, in a case of calculating the influence degree of the systolic pressure (1) with respect to the postoperative infection using the Pearson correlation coefficient, as illustrated in FIG. 29A, the influence degree calculation function 154 calculates the Pearson correlation coefficient by applying, to the following expression (1), x=(162, 154, 126, 146, 110, 122, 103, 128) as numeric values of the explanatory variables of “systolic pressure (1)” and y=(1, 1, 1, 1, 0, 0, 0, 0) obtained by converting “present/absent” of the response variables “postoperative infection” into “1/0”.

$$r = \frac{{\sum }_{i = 1}^{n} ( {x}_{i} - \overline{x} ) ( {y}_{i} - \overline{y} )}{{( ( {\sum }_{i = 1}^{n} {( {x}_{i} - \overline{x} )}^{2} ) ( {\sum }_{i = 1}^{n} {( {y}_{i} - \overline{y} )}^{2} ) )}^{1 / 2}} \qquad ( 1 )$$

For example, when x and y described above are applied to the expression (1), the Pearson correlation coefficient “r” is “62.5/78.2=0.80”. For example, the influence degree calculation function 154 calculates the influence degree of the systolic pressure (1) with respect to the postoperative infection to be “0.80” For example, in a case of calculating the influence degree of the systolic pressure (1) with respect to the postoperative infection using the standard partial regression coefficient, as illustrated in FIG. 29B, the influence degree calculation function 154 calculates the standard partial regression coefficient by applying, to the following expression (2), x1=(162, 154, 126, 146, 110, 122, 103, 128) as numeric values of the explanatory variables of “systolic pressure (1)”, x2=(1, 1, 0, 1, 1, 0, 0, 1) obtained by converting “present/absent” of the explanatory variables of “ascites” into “1/0”, and y=(1, 1, 1, 1, 0, 0, 0, 0) obtained by converting “present/absent” of the response variables of “postoperative infection” into “1/0”.

$$β = \frac{{r}_{x 1 y} - ( {r}_{x 2 y} \times {r}_{x 1 x 2} )}{1 - {r}_{x 1 x 2}^{2}} \qquad ( 2 )$$

In the expression (2), “rx1y” represents a correlation coefficient of y and x1, “rx2y” represents a correlation coefficient of y and x2, and “rx1x2” represents a correlation coefficient of x1 and x2. For example, x1, x2, and y described above are applied to the expression (2), the partial regression coefficient “β” is “0.80−(0.26×0.57)/1−(0.57)2=0.97”. For example, the influence degree calculation function 154 calculates the influence degree of the systolic pressure (1) with respect to the postoperative infection to be “0.97”.

The example described above is merely an example, and the embodiment is not limited thereto. That is, a method of calculating the influence degree by the influence degree calculation function 154 is optional. The influence degree can be calculated by using other various methods that enable the influence degree (for example, correlation) to be calculated.

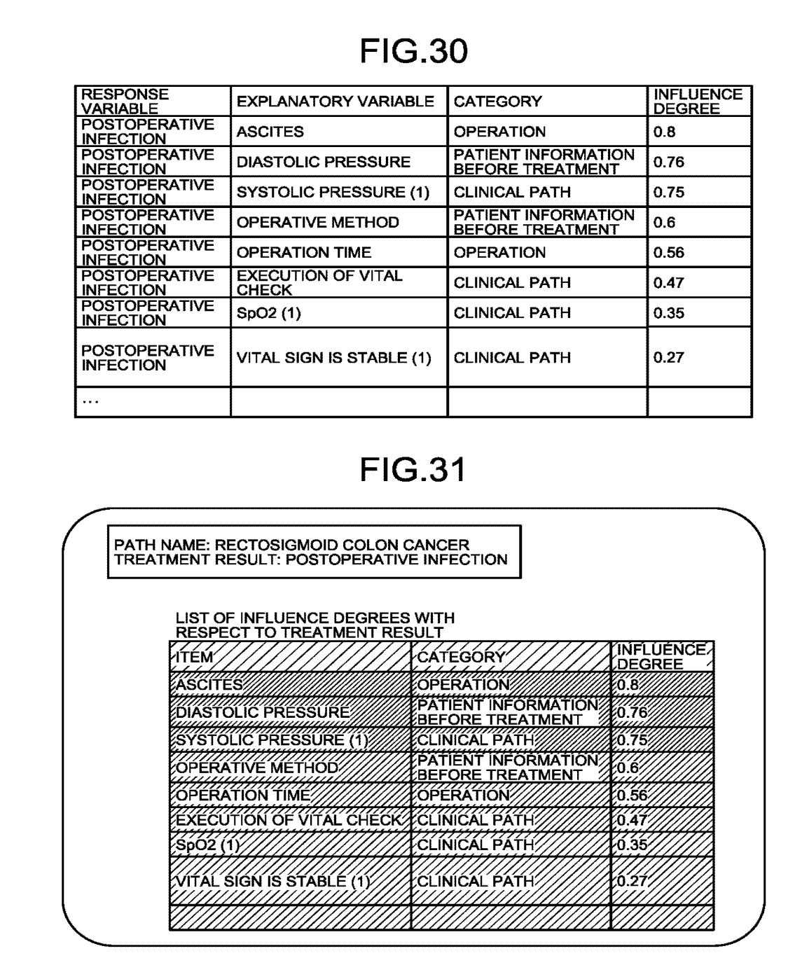

The influence degree calculation function 154 calculates the influence degree of each explanatory variable (each item) with respect to the designated response variable (treatment result), and outputs the calculated influence degree to the display control function 155. FIG. 30 is a diagram illustrating an example of a calculation result of the influence degrees obtained by the influence degree calculation function 154 according to the first embodiment. For example, as illustrated in FIG. 30, the influence degree calculation function 154 calculates the influence degree of each explanatory variable “each item” with respect to “response variable: postoperative infection”.

Returning to FIG. 1, the display control function 155 presents the influence degree. FIG. 31 is a diagram illustrating an example of display of the influence degrees performed by the display control function 155 according to the first embodiment. For example, as illustrated in FIG. 31, the display control function 155 displays a list of influence degrees with respect to the treatment results together with “path name: rectosigmoid colon cancer” and “treatment result: postoperative infection” designated by the user. For example, as illustrated in FIG. 31, the display control function 155 arranges the influence degrees of the respective items in descending order to be displayed by the display 140. For example, the display control function 155 causes the influence degrees to be color-coded and displayed for each range of the influence degrees. For example, the display control function 155 causes the display 140 to display an influence degree list in which the item having the influence degree “equal to or larger than 0.70” is shown in red, the item having the influence degree “equal to or larger than 0.4 and smaller than 0.7” is shown in yellow, and the item having the influence degree “smaller than 0.4” is shown in green.

The processing functions included in the processing circuitry 150 have been described above. Each of the processing functions described above is, for example, stored in the storage 120 as a computer-executable program. The processing circuitry 150 reads out each program from the storage 120 and executes the read program to implement a processing function corresponding to the program. In other words, the processing circuitry 150 that has read out each program has each processing function illustrated in FIG. 1.

FIG. 1 illustrates the example in which each of the processing functions described above is implemented by the single processing circuitry 150, but the embodiment is not limited thereto. For example, the processing circuitry 150 may be configured by combining a plurality of independent processors, and may implement each processing function when each processor executes the program. The processing functions included in the processing circuitry 150 may be appropriately distributed to or integrated with a single processing circuit or a plurality of processing circuits.

The word “processor” used in the above description means, for example, a central processing unit (CPU), a graphics processing unit (GPU), or a circuit such as an application specific integrated circuit (ASIC) and a programmable logic device (for example, a simple programmable logic device (SPLD), a complex programmable logic device (CPLD), and a field programmable gate array (FPGA)). The processor reads out and executes the program stored in the storage 120 to implement the function. Instead of storing the program in the storage 120, the program may be configured to be directly incorporated into the circuit of the processor. In this case, the processor reads out and executes the program incorporated into the circuit to implement the function. Each processor according to the embodiment is not necessarily configured as a single circuit for each processor. A plurality of independent circuits may be combined to be one processor to implement the function.

The program to be executed by the processor is incorporated into a read only memory (ROM), the storage 120, and the like in advance to be provided. The program may be recorded and provided, as a file installable or executable in these devices in a computer-readable storage medium such as a compact disc read only memory (CD-ROM), a flexible disk (FD), a compact disc recordable (CD-R), and a digital versatile disc (DVD). The program may be stored in a computer connected to a network such as the Internet and provided or distributed by being downloaded via the network. For example, the program is configured by a module including functional parts described later. As actual hardware, when the CPU reads out the program from a storage medium such as a ROM to be executed, each module is loaded into a main storage device to be generated on the main storage device.

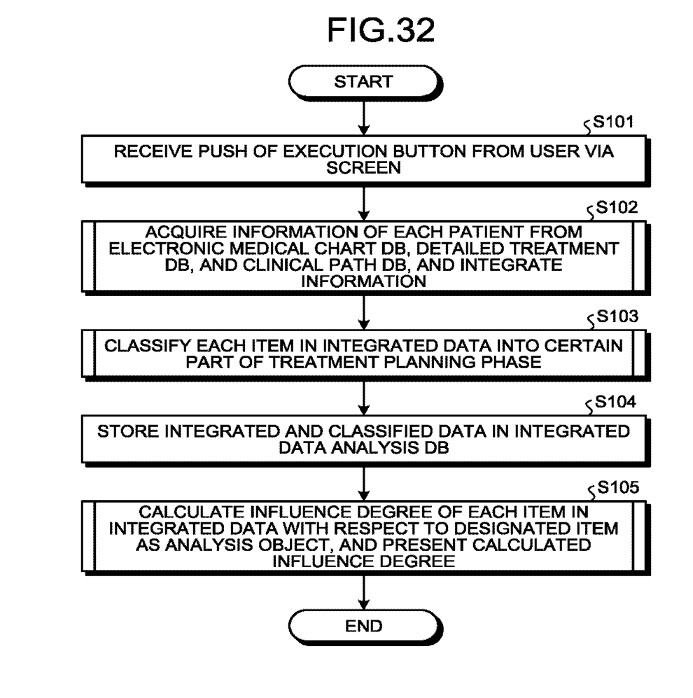

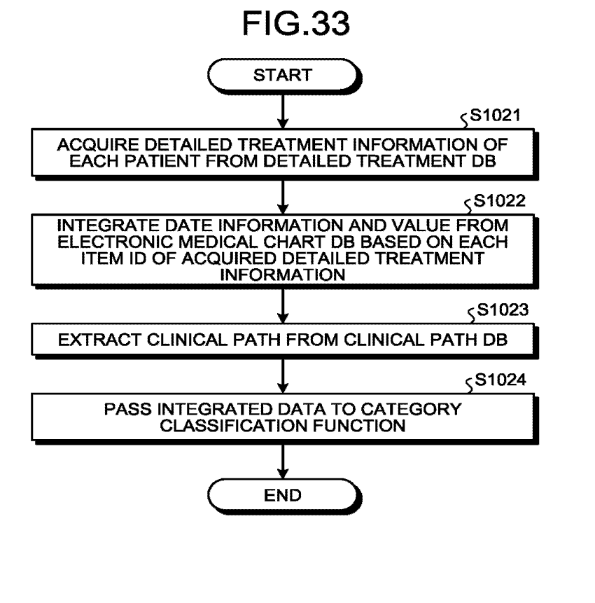

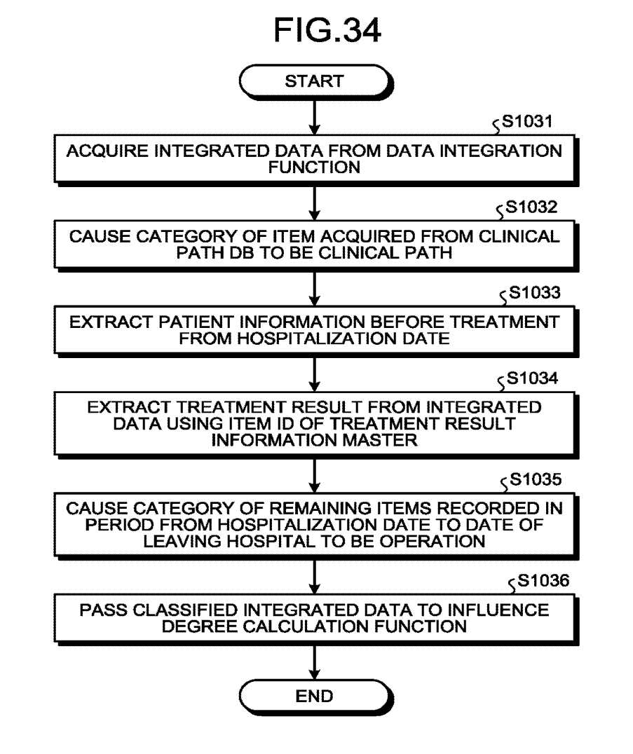

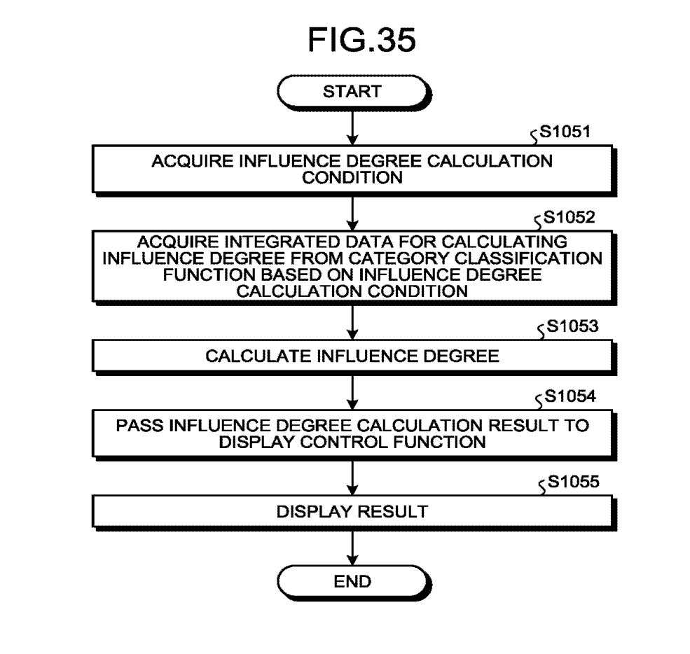

Next, the following describes a procedure of processing performed by the medical information processing apparatus 100 according to the first embodiment with reference to FIGS. 32 to 35. FIGS. 32 to 35 are flowcharts illustrating a procedure of processing performed by the medical information processing apparatus 100 according to the first embodiment. FIG. 33 illustrates details about the processing at Step S102 in FIG. 32. FIG. 34 illustrates details about the processing at Step S103 in FIG. 32. FIG. 35 illustrates details about the processing at Step S105 in FIG. 32.

Herein, Step S101 in FIG. 32 is a step performed by the input interface 130. Step S102 is a step at which the processing circuitry 150 reads out a program corresponding to the data integration function 152 from the storage 120 to be executed. Steps S103 and S104 are steps at which the processing circuitry 150 reads out a program corresponding to the category classification function 153 from the storage 120 to be executed. Step S105 is a step at which the processing circuitry 150 reads out a program corresponding to the influence degree calculation function 154 and the display control function 155 from the storage 120 to be executed.

As illustrated in FIG. 32, at Step S101, the input interface 130 receives a push of an execution button from the user via a screen. At Step S102, the processing circuitry 150 acquires information about each patient from the electronic medical chart storage apparatus (electronic medical chart DB), the detailed treatment information storage apparatus (detailed treatment DB), and the electronic medical chart storage apparatus (clinical path DB), and integrates the information.

At Step S103, the processing circuitry 150 classifies each item in the integrated data into a certain part of a treatment planning phase. That is, the processing circuitry 150 classifies each item into a certain category in a course of treatment period. At Step S104, the processing circuitry 150 stores the integrated and classified data in the storage 120 (integrated data analysis DB).

Next, at Step S105, the processing circuitry 150 calculates the influence degree of each item of the integrated data with respect to the designated item as an analysis object, and presents the calculated influence degree.

As illustrated in FIG. 33, at Step S1021, the processing circuitry 150 acquires detailed treatment information of each patient from the detailed treatment DB. At Step S1022, the processing circuitry 150 integrates date information and a value from the electronic medical chart DB based on each item ID of the acquired detailed treatment information. At Step S1023, the processing circuitry 150 extracts the clinical path from the clinical path DB. At Step S1024, the data integration function 152 of the processing circuitry 150 passes the integrated data to the category classification function 153.

As illustrated in FIG. 34, at Step S1031, the category classification function 153 of the processing circuitry 150 acquires the integrated data from the data integration function 152. At Step S1032, the processing circuitry 150 causes the category of the item acquired from the clinical path DB to be the clinical path. At Step S1033, the processing circuitry 150 acquires the patient information before treatment from the hospitalization date.

At Step S1034, the processing circuitry 150 extracts a treatment result from the integrated data using the item ID of treatment result information master table. At Step S1035, the processing circuitry 150 causes the category of remaining items recorded in a period from the hospitalization date to the date of leaving the hospital to be the operation. Subsequently, at Step S1036, the category classification function 153 of the processing circuitry 150 passes the classified integrated data to the influence degree calculation function 154.

As illustrated in FIG. 35, at Step S1051, the processing circuitry 150 acquires an influence degree calculation condition. At Step S1052, the influence degree calculation function 154 of the processing circuitry 150 acquires the integrated data for calculating the influence degree from the category classification function 153 based on the influence degree calculation condition. At Step S1053, the processing circuitry 150 calculates the influence degree. At Step S1054, the influence degree calculation function 154 of the processing circuitry 150 passes the influence degree calculation result to the display control function 155. At Step S1055, the processing circuitry 150 displays a result.

As described above, according to the first embodiment, the data integration function 152 generates integrated data obtained by integrating the information before and after the period to which the clinical path is applied (information outside the hospitalization period) and the information during a period to which the clinical path is applied (information during the hospitalization period). The category classification function 153 classifies the information included in the integrated data into a plurality of categories based on a corresponding period and type. The influence degree calculation function 154 calculates the influence degree of each piece of information included in a plurality of categories with respect to the designated item as an analysis object in the information included in the integrated data. The display control function 155 presents the influence degree. Accordingly, the medical information processing apparatus 100 according to the first embodiment enables a variance to be analyzed by using total information of treatment.

For example, presently, importance is attached to improvement of a treatment process and improvement in quality of healthcare by using the clinical path to standardize a medical care plan. To improve the quality of healthcare using the clinical path, a Plan-Do-Check-Act (PDCA) cycle is considered to be important, the PDCA cycle of collecting and analyzing a variance as a difference between the clinical path and actual medical care, and continuously coping with a factor of the variance that affects the quality of healthcare.

However, in the related art, analysis is made by using only information associated with the clinical path, so that it has been difficult to correctly analyze a variance caused by information unassociated with the clinical path. For example, among factors of the variance caused by a staff or a system, some factors are obvious from a situation at the time when the variance is generated, but the factor caused by a patient such as retardation of recovery due to a complication is often not obvious only from the situation at the time when the variance is generated.

With the medical information processing apparatus 100 according to the first embodiment, analysis can be made in consideration of information unassociated with the clinical path by analyzing the influence degree of each item using the total treatment information. That is, the medical information processing apparatus 100 enables various objects that have been unanalyzable to be analyzed. For example, the medical information processing apparatus 100 can make analysis in consideration of data that is recorded before the clinical path is applied (for example, a determined operative method and an inspection value), and according to a result thereof, the user can correct an application condition of the clinical path.

For example, a variance of a ruptured suture in surgery may be influenced not only by the item included in the clinical path but also a detailed item of the surgery (example: blood transfusion before surgery) such as a case report. Even in such a case, the medical information processing apparatus 100 according to the first embodiment can make analysis more correctly.

According to the first embodiment, the category classification function 153 classifies the information included in the integrated data into categories of the patient information before treatment, the information about an operation, the information about the clinical path, and the information about a treatment result. The influence degree calculation function 154 calculates the influence degrees of the patient information before treatment, the information about an operation, and the information about the clinical path with respect to the designated item as an analysis object in the information about a treatment result. Accordingly, the medical information processing apparatus 100 according to the first embodiment enables analysis to be made in accordance with various purposes. For example, the medical information processing apparatus 100 enables the treatment result to be analyzed from various viewpoints.

According to the first embodiment, the data integration function 152 acquires information in a predetermined period before and after the period to which the clinical path is applied. The data integration function 152 generates the integrated data that is integrated based on an application date of the clinical path. Thus, the medical information processing apparatus 100 according to the first embodiment can correctly integrate the information within the period to which the clinical path is applied and the information outside the period to which the clinical path is applied. For example, a case report such as inspection data and operation recording data is not stored in consideration of the clinical path, and if they are simply integrated with the clinical path, analysis cannot be made correctly. By way of example, the inspection value of preoperative information in the case report is in a format of “recording the latest period within 30 days”, so that recorded items do not include accurate date information. Thus, it cannot be determined whether such a value is stored within a range to which the clinical path is applied or recorded outside the range. That is, in a case of analyzing a relation to the item included in the clinical path, analysis cannot be made correctly.

For example, it is assumed that an operative method of “enlarged lymph node dissection” is described in a case report of reflux esophagitis. If the operative method of “enlarged lymph node dissection” is determined before applying the path, the operative method of “enlarged lymph node dissection” can be used as an application condition analysis item of the path. However, in a case in which the operative method of “enlarged lymph node dissection” is recorded during a path application period due to a change of the operative method and the like, the operative method is not used as the application condition analysis item of the path.

Thus, by integrating the data based on the period to which the clinical path is applied (for example, hospitalization date), the medical information processing apparatus 100 can correctly associate the items, and can make correct analysis. In this way, by integrating the data based on the period to which the clinical path is applied, even when the same items are included, the items can be discriminated based on the number of days, so that each of the same items can be correctly analyzed.

The display control function 155 presents corresponding items in a descending order of the influence degree. Thus, the medical information processing apparatus 100 according to the first embodiment enables an item having a high influence degree to be immediately determined.

### Second Embodiment

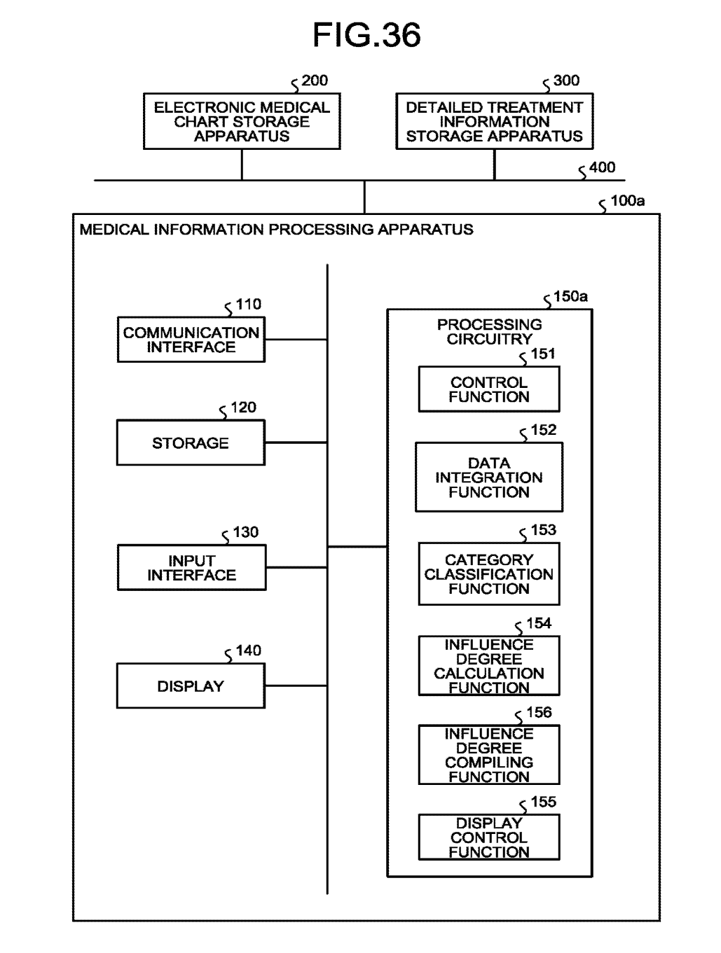

In the first embodiment, described is a case of calculating the influence degree for each item. In a second embodiment, described is a case of calculating the influence degree for each category. FIG. 36 is a diagram illustrating an example of a configuration of a medical information processing apparatus according to the second embodiment. A medical information processing apparatus 100a according to the second embodiment is different from the medical information processing apparatus 100 according to the first embodiment in that processing circuitry 150a executes an influence degree compiling function 156. The following mainly describes the difference therebetween. The same component as that in the first embodiment is denoted by the same reference numeral, and redundant description will not be repeated. The influence degree compiling function 156 according to the second embodiment is an example of a calculation unit in claims.

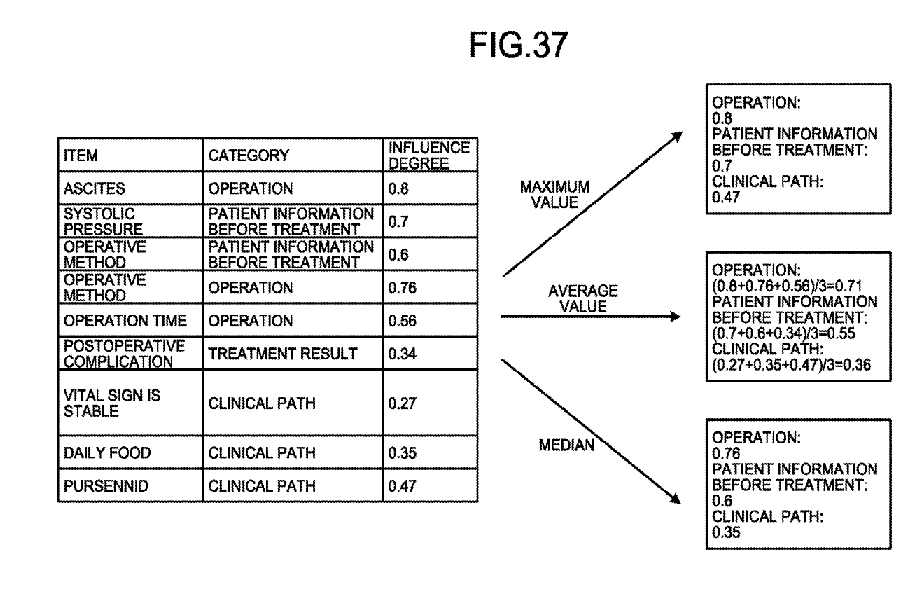

The influence degree compiling function 156 compiles the influence degrees of the pieces of information for each category, and further calculates the influence degree for each category. FIG. 37 is a diagram illustrating an example of compilation of the influence degrees performed by the influence degree compiling function 156 according to the second embodiment. FIG. 37 illustrates processing performed by the influence degree compiling function 156 after the influence degree calculation function 154 calculates the influence degree for each item. For example, as illustrated in FIG. 37, the influence degree compiling function 156 compiles, for each category, the influence degree for each item calculated by the influence degree calculation function 154.

For example, the influence degree compiling function 156 extracts influence degrees of items corresponding to the category of “operation” from among the items, and calculates a maximum value, an average value, and a median. For example, the influence degree compiling function 156 extracts influence degrees of items corresponding to the category of “patient information before treatment” from among the items, and calculates a maximum value, an average value, and a median. For example, the influence degree compiling function 156 extracts influence degrees of items corresponding to the category of “clinical path” from among the items, and calculates a maximum value, an average value, and a median.

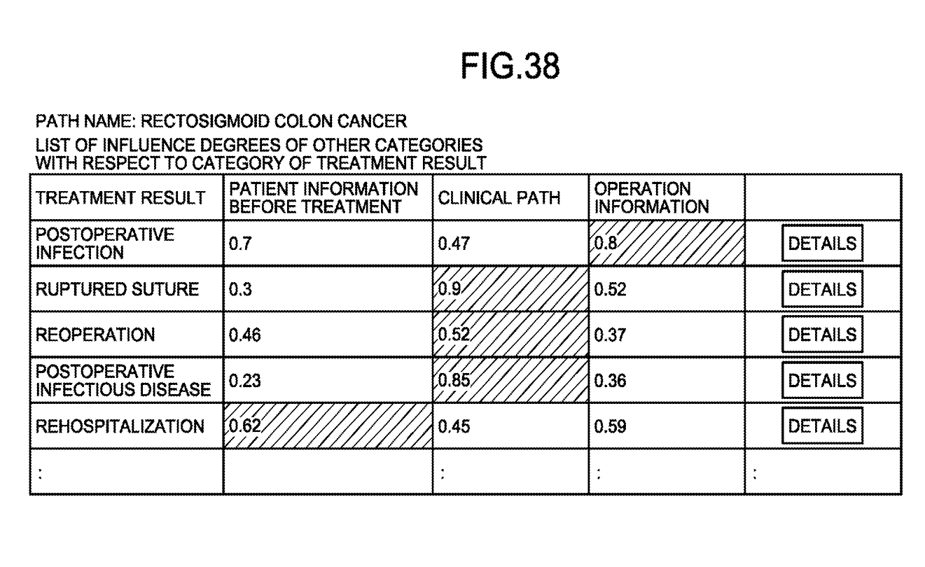

The display control function 155 according to the second embodiment causes the display 140 to display the influence degree for each category compiled by the influence degree compiling function 156. FIG. 38 is a diagram illustrating an example of display of the influence degrees performed by the display control function 155 according to the second embodiment. For example, as illustrated in FIG. 38, the display control function 155 causes the display 140 to display display information indicating the influence degree of each category for each treatment result. By way of example, regarding the path name of “rectosigmoid colon cancer” of the clinical path, the display control function 155 causes the display 140 to display the influence degrees of three categories including “patient information before treatment”, “clinical path”, and “operation” with respect to each treatment result such as “postoperative infection”, “ruptured suture”, “reoperation”, “postoperative infection”, and “rehospitalization”.

The display control function 155 controls the category having the largest influence degree with respect to each treatment result to be enhanced and displayed. For example, as illustrated in FIG. 38, the display control function 155 causes the category of “operation” having the largest influence degree with respect to “postoperative infection” to be enhanced and displayed. When receiving a push of “details” button illustrated in FIG. 38, the display control function 155 can cause detailed information about the influence degree with respect to the treatment result corresponding to the pushed button to be displayed. For example, when the user pushes the “details” button associated with “postoperative infection” via the input interface 130, the display control function 155 causes the display 140 to display the influence degree for each category item that is calculated with respect to “postoperative infection”. Due to this, the user can take an overview of whether an intended improvement effect can be obtained for each treatment result, and can easily check detailed influence degrees.

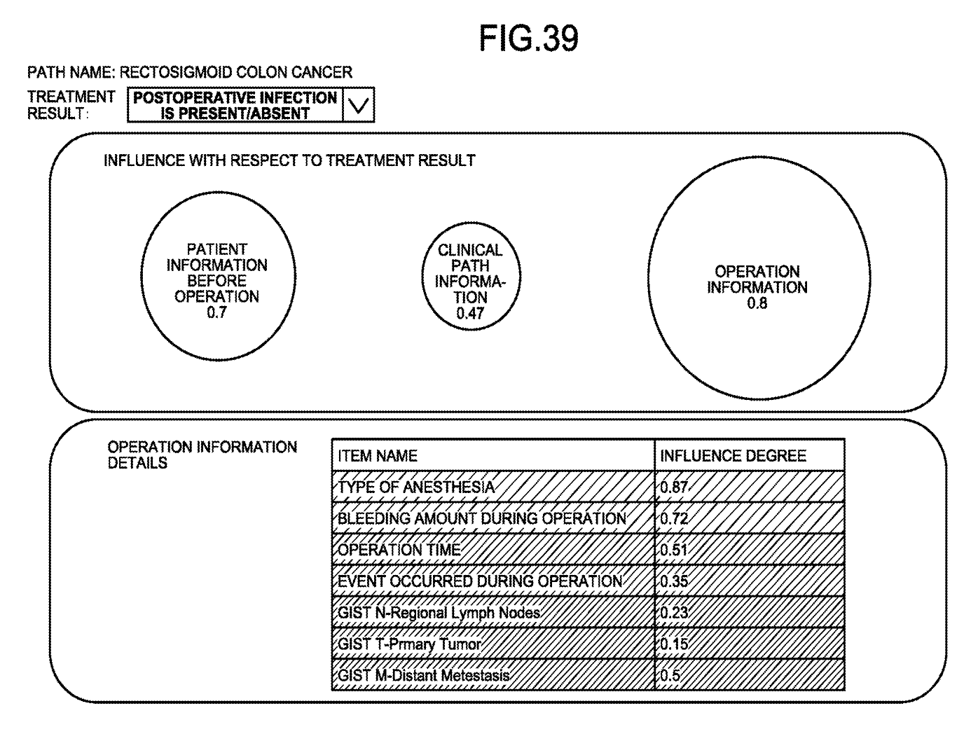

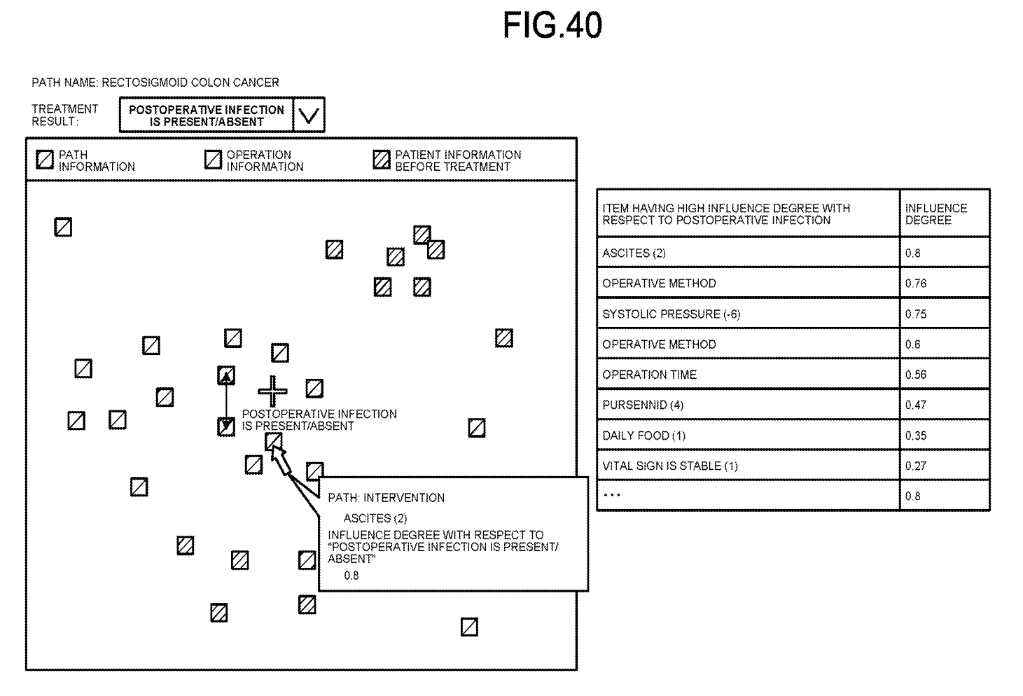

The display example illustrated in FIG. 38 is merely an example, and display of the influence degree performed by the display control function 155 is not limited thereto. The following describes an example of display of the influence degrees performed by the display control function 155 with reference to FIGS. 39 and 40. FIGS. 39 and 40 are diagrams illustrating an example of display of the influence degrees performed by the display control function 155 according to the second embodiment. For example, as illustrated in an upper row of FIG. 39, the display control function 155 causes the display 140 to display the display information indicating the influence degree for each category as a circle. The display control function 155 causes the information representing a difference of the influence degree for each category by a size of the circle to be displayed.

By way of example, regarding the path name of “rectosigmoid colon cancer” of the clinical path, the display control function 155 causes the display information in which a circle indicating the influence degree of “operation” is the largest to be displayed. When receiving a designating operation for each circle illustrated in FIG. 39, the display control function 155 can cause the information about the influence degree of each item of the category corresponding to the designated circle to be displayed. For example, when receiving the designating operation for “operation” from the user via the input interface 130, the display control function 155 causes the display 140 to display the influence degree of each item included in the category of “operation”. The display control function 155 selects the item having a high influence degree in the designated category to be displayed.

For example, as illustrated in FIG. 40, the display control function 155 presents, as a distance, the influence degree between the items regarding the designated path and treatment result, and displays display information for determining whether there is a correlation between the items having a high influence degree. In the display information illustrated in FIG. 40, for example, a center cross corresponds to the treatment result of “postoperative infection is present/absent”, and a plot closer to the center has a higher influence degree (higher correlation). In FIG. 40, the correlation between the items is also indicated by the distance between plots (between the items). A two-dimensional plot illustrated in FIG. 40 can be implemented by using a multidimensional scaling method.

For example, the user can recognize that the clinical path has the highest correlation (highest influence degree) with the treatment result of “postoperative infection is present/absent” with reference to the display information illustrated in FIG. 40. As illustrated in FIG. 40, by superimposing a pointer illustrated as an arrow on each plot indicating the item via the input interface 130, the user can cause detailed information of the plot to be displayed. As illustrated in a right diagram of FIG. 40, the display control function 155 can cause the item having a high influence degree with respect to the designated treatment result of “postoperative infection is present/absent” to be displayed in descending order together with the two-dimensional plot.

As described above, according to the second embodiment, the influence degree compiling function 156 complies the influence degree of each piece of information for each category, and further calculates the influence degree for each category. Accordingly, the medical information processing apparatus 100a according to the second embodiment can display the influence degree for each category, the influence degree for each item, and the influence degree for each execution date of the item in a stepwise manner. Due to this, the medical information processing apparatus 100a enables the influence degree with respect to the treatment object to be analyzed from various viewpoints.

### Third Embodiment

Although the first and the second embodiments have been described above, various other embodiments may be used in addition to the first and the second embodiments described above.

In the above embodiments, described is a case of integrating the information during the hospitalization period and the information outside the hospitalization period to be analyzed. However, the embodiment is not limited thereto. For example, the information of the treatment execution date and the information outside the treatment execution date may be integrated to be analyzed. In such a case, for example, the medical information processing apparatus and the medical information processing method according to the present application can be applied to an outpatient operation, outpatient radiation treatment, and the like.

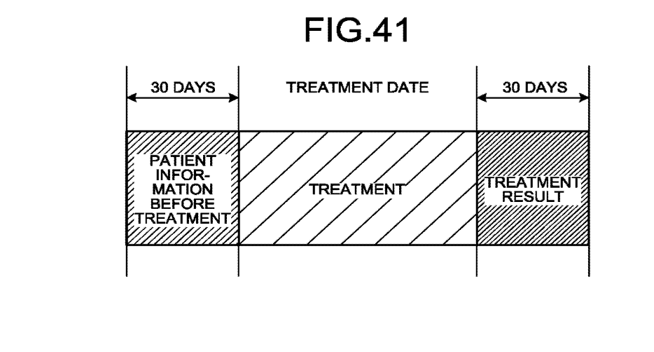

FIG. 41 is a diagram for explaining analysis content according to a third embodiment. For example, as illustrated in FIG. 41, the medical information processing apparatus 100 according to the present embodiment uses the information of the treatment execution date at which treatment is performed, the patient information before treatment 30 days before the treatment execution date, and the treatment result 30 days after the treatment execution date to generate the integrated data, and makes analysis using the generated integrated data. The period outside the treatment execution date is not limited to 30 days illustrated in the drawing, and any period can be used.

In a case of making analysis by integrating the information of the treatment execution date and the information outside the treatment execution date, a plan of medical practice executed at the treatment execution date (execution plan of the treatment execution date) corresponds to the clinical path described above. The execution plan of the treatment execution date is, for example, vital check. That is, the control function 151 according to the present embodiment acquires various pieces of data and information related to the execution plan of the treatment execution date corresponding to various pieces of data and information related to the clinical path described in the first and the second embodiments, and causes the storage 120 to store the pieces of data and information.

The data integration function 152 according to the present embodiment generates integrated data obtained by integrating the information of the treatment execution date and the information outside the treatment execution date. Specifically, the data integration function 152 generates the integrated data obtained by integrating information associated with the execution plan of the treatment execution date and information unassociated with the execution plan of the treatment execution date. That is, the data integration function 152 generates the integrated data indicating total treatment information of the patient. For example, similarly to the case of using the data related to the clinical path described above, the data integration function 152 generates the integrated data obtained by integrating the information of the treatment execution date and the information outside the treatment execution date, and causes the storage 120 to store the generated integrated data. The data integration function 152 generates the integrated data based on the treatment execution date.

The category classification function 153 according to the present embodiment classifies the information included in the integrated data into a plurality of categories based on a corresponding period and type. Specifically, the category classification function 153 classifies the integrated data stored by the storage 120 into a plurality of categories.

For example, the category classification function 153 classifies the information included in the integrated data into categories of patient information before treatment, information about treatment (for example, information about an operation or radiation treatment), information about the execution plan of the treatment execution date, and information about a treatment result.

The influence degree calculation function 154 according to the present embodiment then calculates the influence degree of the information of the integrated data included in corresponding one of the categories related to the designated item as an analysis object of the patient with respect to the item. Specifically, the influence degree calculation function 154 calculates the influence degree of each item included in each category with respect to the information as an analysis object received via the input interface 130. For example, the influence degree calculation function 154 calculates respective influence degrees of the patient information before treatment, the information about treatment, and the information about the execution plan of the treatment execution date with respect to the designated item as an analysis object in the information about the treatment result. As a method of designating the analysis object as an object of influence degree calculation, various methods can be used similarly to the first and the second embodiments described above.

The display control function 155 according to the present embodiment then presents the influence degree. The display control function 155 according to the present embodiment can variously perform display similarly to the first and the second embodiments described above.

In the example described above, described is a case in which the treatment execution date is one day. However, the embodiment is not limited thereto. For example, a period required for outpatient radiation treatment can be set as a treatment execution date. In such a case, the integrated data is generated by using information about an execution plan of medical practice during a period of radiation treatment that is planned for a certain period and information about a plurality of times of radiation treatment.

In this way, the medical information processing apparatus 100 according to the present embodiment can analyze a variance not only by using the information during the hospitalization period and the information outside the hospitalization period, but also by using total information of treatment in an outpatient operation, outpatient radiation treatment, or the like. That is, the medical information processing apparatus 100 can analyze a cause of a difference between the execution plan (medical care plan) of the treatment execution date and actual medical care by using the total information of treatment.

For example, the components of the devices illustrated in the drawings according to the embodiments described above are merely conceptual, and it is not required that it is physically configured as illustrated necessarily. That is, specific forms of distribution and integration of the devices are not limited to those illustrated in the drawings. All or part thereof may be functionally or physically distributed/integrated in arbitrary units depending on various loads or usage states. All or any part of the processing functions executed by the devices may be implemented by a CPU or a program that is analyzed and executed by the CPU, or may be implemented as hardware based on wired logic.

According to at least one of the embodiments described above, a variance can be analyzed by using total information of treatment.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus comprising processing circuitry configured to:
generate integrated data obtained by integrating information outside a hospitalization period and information during the hospitalization period;
classify information included in the integrated data into categories based on a period and a type; and
calculate an influence degree of information of the integrated data included in corresponding one of the categories related to a designated item as an analysis object of a patient with respect to the item.

2. The medical information processing apparatus according to claim 1, wherein the processing circuitry is configured to
classify the information included in the integrated data into categories of patient information before treatment, information about treatment, information about a clinical path, and information about a treatment result, and
calculate respective influence degrees of the patient information before treatment, the information about treatment, and the information about the clinical path with respect to the designated item as the analysis object in the information about the treatment result.

3. The medical information processing apparatus according to claim 1, wherein the processing circuitry is configured to compile the influence degrees of pieces of the information for each of the categories, and further calculate the influence degree for each category.

4. The medical information processing apparatus according to claim 1, wherein the processing circuitry is configured to acquire information in a predetermined period outside the hospitalization period.

5. The medical information processing apparatus according to claim 1, wherein the processing circuitry is configured to generate integrated data that is integrated based on a hospitalization date.

6. The medical information processing apparatus according to claim 1, wherein the processing circuitry is configured to present corresponding information in descending order of the influence degree.

7. The medical information processing apparatus according to claim 1, wherein the processing circuitry is configured to enhance and present a category having a high influence degree with respect to the item as the analysis object.

8. The medical information processing apparatus according to claim 1, wherein the processing circuitry is configured to present display information indicating a difference in the influence degree with respect to the item as the analysis object as a size of a displayed object.

9. The medical information processing apparatus according to claim 1, wherein the processing circuitry is configured to present display information indicating a difference in the influence degree with respect to the item as the analysis object as a distance between displayed objects.

10. A medical information processing apparatus comprising processing circuitry configured to:
generate integrated data obtained by integrating information outside a treatment execution date and information of the treatment execution date;
classify information included in the integrated data into categories based on a period and a type; and
calculate an influence degree of information of the integrated data included in corresponding one of the categories related to a designated item as an analysis object of a patient with respect to the item.

11. The medical information processing apparatus according to claim 10, wherein the processing circuitry is configured to
classify the information included in the integrated data into categories of patient information before treatment, information about treatment, information about an execution plan of the treatment execution date, and information about a treatment result, and
calculate respective influence degrees of the patient information before treatment, the information about treatment, and the information about the execution plan of the treatment execution date with respect to the designated item as the analysis object in the information about a treatment result.

12. The medical information processing apparatus according to claim 10, wherein the processing circuitry is configured to compile the influence degrees of pieces of the information for each of the categories, and further calculate the influence degree for each category.

13. The medical information processing apparatus according to claim 10, wherein the processing circuitry is configured to acquire information in a predetermined period outside the treatment execution date.

14. The medical information processing apparatus according to claim 10, wherein the processing circuitry is configured to generate integrated data that is integrated based on the treatment execution date.

15. The medical information processing apparatus according to claim 10, wherein the processing circuitry is configured to present corresponding information in descending order of the influence degree.

16. The medical information processing apparatus according to claim 10, wherein the processing circuitry is configured to enhance and present a category having a high influence degree with respect to the item as the analysis object.

17. The medical information processing apparatus according to claim 10, wherein the processing circuitry is configured to present display information indicating a difference in the influence degree with respect to the item as the analysis object as a size of a displayed object.

18. The medical information processing apparatus according to claim 10, wherein the processing circuitry is configured to present display information indicating a difference in the influence degree with respect to the item as the analysis object as a distance between displayed objects.

19. A medical information processing method comprising:
generating integrated data obtained by integrating information outside a hospitalization period and information during the hospitalization period;
classifying information included in the integrated data into categories based on a period and a type; and
calculating an influence degree of information of the integrated data included in corresponding one of the categories related to a designated item as an analysis object of a patient with respect to the item.

20. A medical information processing method comprising:
generating integrated data obtained by integrating information outside a treatment execution date and information of the treatment execution date;
classifying information included in the integrated data into categories based on a period and a type; and
calculating an influence degree of information of the integrated data included in corresponding one of the categories related to a designated item as an analysis object of a patient with respect to the item.
